(19) United States
(12) Patent Application Publication
(10) Pub. No.: US 2024/0075088 A1
(43) Pub. Date: Mar. 7, 2024

(54) BIOACTIVE EXTRACTION METHOD

(71) Applicant: Performance Labs Pte. Ltd., Singapore (SG)

(72) Inventor: Dan Li, Singapore (SG)

(21) Appl. No.: 18/458,420

(22) Filed: Aug. 30, 2023

Related U.S. Application Data
(60) Provisional application No. 63/374,353, filed on Sep. 1, 2022.

(51) Int. Cl. A61K 36/185; A61K 31/522

(52) U.S. Cl. CPC A61K 36/185; A61K 31/522; A61K 2236/331; A61K 2236/333; A61K 2236/53

(57) ABSTRACT

The present disclosure generally relates to compositions of decaffeinated caffeoylquinic acid botanical extracts, and to methods of removing extracting a bioactive compound from a plant material. In some embodiments, the methods include obtaining a plant material; mixing the plant material with a natural deep eutectic solvent (NaDES); obtaining a crude extract solution comprising extracts of the plant material, the bioactive compound, and NaDES; loading the crude extract solution onto a macroporous resin; and recovering plant extract from the macroporous resin, wherein the bioactive compound and NaDES have been removed from the plant extract. In some embodiments, the bioactive compound is caffeine. In some embodiments, the plant material is obtained from yerba mate.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims priority to U.S. Provisional Application No. 63/374,353, filed Sep. 1, 2022, and entitled “BIOACTIVE EXTRACTION METHOD,” which is hereby incorporated by reference in its entirety.

### FIELD

The present disclosure relates generally to the field of plant extraction processes. In particular, the disclosure relates to methods for extracting bioactive compounds, such as caffeine, from a botanical, such as yerba mate. The methods include extracting with deep eutectic solvents combined with macroporous adsorption resin chromatography.

### BACKGROUND

Ilex paraguariensis (yerba mate) is a medicinal plant that includes numerous bioactive compounds, including caffeine, chlorogenic acid, saponins, and xanthine alkaloids. Traditional extraction of these compounds from yerba mate uses harsh processes and chemicals that are not environmentally friendly or efficient.

Natural deep eutectic solvents (NaDESs) are ubiquitous compounds that solubilize various metabolites and compounds, and are environmentally friendly.

Macroporous adsorption resins (MARs) are synthetic porous crosslinked polymer beads that separate and purify pharmaceutical and natural products.

### SUMMARY

The present disclosure generally relates to compositions of decaffeinated caffeoylquinic acid botanical extracts, and methods of removing caffeine from botanicals using natural deep eutectic solvents in combination with microporous resin adsorption chromatography.

Some embodiments provided herein relate to methods of extracting a bioactive compound from a plant material. In some embodiments, the methods include obtaining a plant material. In some embodiments, the methods further include mixing the plant material with a natural deep eutectic solvent (NaDES). In some embodiments, the methods further include obtaining a crude extract solution comprising extracts of the plant material, the bioactive compound, and NaDES. In some embodiments, the methods further include loading the crude extract solution onto a macroporous resin. In some embodiments, the method further includes recovering plant extract from the macroporous resin, wherein the bioactive compound and NaDES have been removed from the plant extract. In some embodiments, the bioactive compound is caffeine. In some embodiments, the plant material is obtained from yerba mate.

In some embodiments, the NaDES includes choline chloride (ChCl) and glucose (GLU). In some embodiments, the choline chloride and glucose are present in a ratio of about 2:1. In some embodiments, the NaDES includes water present in an amount ranging from 10%-50% by weight. In some embodiments, the NaDES includes water present in an amount of about 30% by weight.

In some embodiments, the methods also include diluting the crude extract with water before applying the crude extract to the macroporous resin. In some embodiments, the dilution is about a 10× dilution.

In some embodiments, the recovered plant extract is enriched for caffeoylquinic acids. In some embodiments, the recovered plant extract contains less than 0.05% caffeine by weight. In some embodiments, the recovered plant extract contains less than 0.01% caffeine by weight.

In some embodiments, the macroporous resin includes a non-polar macroporous resin. In some embodiments, the macroporous resin includes HDP200. In some embodiments, the macroporous resin is integrated into a column.

In some embodiments, recovering the plant extract includes eluting the plant extract from the macroporous resin. In some embodiments, the plant extract is eluted with a solution comprising ethanol in an amount ranging from 10% to 30% by volume. In some embodiments, the solution includes about 20% ethanol by volume.

In some embodiments, the methods also include recovering NaDES from the macroporous resin. In some embodiments, recovering NaDES from the macroporous resin includes washing the macroporous resin with water, capturing effluent and water, and drying the captured effluent and water to recover the NaDES.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIG. 1 illustrates a schematic representation of an embodiment for a process for the recovery of natural deep eutectic solvents and manufacture of decaffeinated yerba mate extracts.

FIG. 2 depicts the biosynthesis of caffeine in tea.

FIG. 3 depicts the chemical structure and properties of caffeine.

FIG. 4 depicts exemplary HPLC chromatograms at 280 nm and 350 nm of yerba mate extracted by ChCl:CA (chlorine chloride:citric acid) solvents in a ratio of 2:1.

FIG. 5 depicts a bar graph showing a comparison of extraction yield of yerba mate by different NaDESs with water and ethanol, including (from left to right): water, CHCL:CA 2:1, CHCL:CA 1:1, CHCL:CA 1:2, CHCL:CA 1:3, CHCL:Glu 2:1, Glu:CA 1:1, and ethanol.

FIGS. 6A-6B depict the static adsorption/desorption capacities and desorption ratio of bioactives in yerba mate on different macroporous resins.

FIGS. 7A-7E depict exemplary HPLC chromatograms of samples obtained in column chromatography process. Each sample contains two chromatograms: 280 nm for theobromine and caffeine; 350 nm for all caffeoylquinic acids and rutin.

FIGS. 8A-8C depict exemplary HPLC chromatograms of samples obtained in column chromatography process. Each sample contain two chromatograms: 280 nm for theobromine and caffeine; 350 nm for all chlorogenic acids and rutin.

FIG. 9 depicts an exemplary line graph showing results of a 2,2-diphenyl-1-picrylhydrazyl (DPPH) antioxidant assay of different fractions.

FIG. 10 illustrates chemical structures of caffeoylquinic acid isomers.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the drawings, can be arranged, substituted, combined, separated, and designed in a wide variety of different configurations, all of which are explicitly contemplated herein.

As summarized herein, aspects of methods for removing and extracting bioactive compounds from plant materials using a combination of natural deep eutectic solvents (NaDESs) and macroporous adsorption resin (MAR) chromatography are provided herein.

It is to be understood that this disclosure is not limited to particular embodiments described, as such may, of course, vary. It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Unless defined otherwise, technical and scientific terms used herein have the same meaning as commonly understood by one of ordinary skill in the art to which the present disclosure belongs. For purposes of the present disclosure, the following terms are defined below.

As used herein, the term “extract”, “extraction”, “extracting”, or any derivative thereof has its ordinary meaning as understood in light of the specification, and refers to a process of removing material, compounds, compositions, or other components from a starting material. A process of extraction can be performed by various means, including by physical or chemical extraction, such as by pressing, grinding, heating, stifling, or other known methods for extracting a component from a starting material.

As used herein, the term “plant” has its ordinary meaning as understood in light of the specification, and refers to a whole plant or any parts or derivatives thereof, such as plant cells, plant protoplasts, plant cell tissue cultures from which plants can be regenerated, plant calli, embryos, pollen, ovules, fruit, flowers, leaves, seeds, roots, root tips, and the like. The term “botanical” is used herein interchangeably with plant, and refers to a plant or any parts derived thereof.

In some embodiments, the plant is any plant having components therein that are desirable to extract, purify, or obtain from the plant. For example, the plant is or is derived from maca, he shou wu, iporuru (Alchornea castaneifolia), kanna (Sceletium Tortosum), honokiol (Magnolia grandiflora), jujube (Ziziphi Spinosae), cnidium (Fructus Cnidii), corydalis (Corydalis yanhusuo), albizia (Cortex albiziae), ginseng (Panax ginseng), polygonum (Polygoni Multiflori), fu ling (Poria cocos), cornus (Fructus corni), Chinese yam (Rhizoma dioscoreae), muira puama, Dendrobium sp., licorice root radix (Glycyrrhizae Preparata), cordyceps (Cordyceps sinensis), Chinese angelica (Angelicae sinensis), kratom (Mitragyna speciosa), bacopa monnieri, catuaba, ashwaghanda, peganum harmala, wheat, alfalfa, oat, kamut, echinacea, chlorella, amla, stinging nettles, carob, mesquite, chuchuhuasai, clavo huasca, chanca piedra, guayusa, rhodiola rosea, shilajit, higenamine, moringa (Moringa oleifera), horny goat weed (Epidmedium), astragalus, aloe vera, turmeric, pine, curcumin (turmeric compound), hops, xanthohumol (hops compound), passion flower, mucuna puriens, tusli, black pepper, bioperine (black pepper compound), Siberian ginseng, American ginseng, yerba mate, lemon balm, astragulus, kava, schizandra, skullcap, valerian, California poppy, epidmedium, pau d'arco, gingko, lotus, lily, tea, coffee, or cacao, or any components of such plants, including bark, leaves, stems, roots, flowers, fruits, pollens, seeds or the like. Plant ingredients may include plant oils, including, for example linalool; b-caryophyllene; b-myrcene; d-limonene; humulene; a-pinene; ylang (Cananga odorata); yarrow (Achillea millefolium); violet (Viola odorata); vetiver (Vetiveria zizanoides); vanilla (Vanilla plantifolia); tuberose (Polianthes tuberosa); thyme (Thymus vulgaris L.); tea tree (Melaleuca alternifolia); tangerine (Citrus reticulata); spruce (Picea mariana); spruce (Tsuga Canadensis); zpikenard (Nardostachys jatamansi); spearmint (Mentha spicata); sandalwood (Santalum spicatum); rosewood (Aniba rosaeodora); rosemary verbenone (Rosmarinus officinalis); rosemary (Rosmarinus officinalis); rose (Rosa damascena); rose geranium (Pelargonium roseum); ravensara (Ravensara aromatica); plai (Zingiber cassumunar); pine needle (Pinus sylvestris L.); petitgrain (Citrus aurantium); peppermint (Mentha piperita); pepper (Piper nigrum L.); patchouli (Pogostemon cablin); palo santo (Bursera graveolens); palmarosa (Cymbopogon martini); osmanthus (Osmanthus fragrans); oregano (Origanum vulgare); orange (Citrus sinensis); oak moss (Evernia prunastri); nutmeg (Myristica fragrans); niaouli (Melaleuca viridifloria); neroli (Citrus aurantium); myrtle (Myrtus communis); myrrh (Commiphora myrrha); mimosa (Acacia decurrens); melissa (Melissa officinalis L.); marjoram (Origanum majorana); manuka (Leptospermum scoparium); mandarin (Citrus deliciosa); mandarin (Citrus deliciosa); lotus (Nelumbo nucifera); lotus (Nelumbo nucifera); lotus (Nelumbo nucifera); lime (Citrus aurantifolia); lily (Lilum aurantum); lemongrass (Cymbopogon citratus); lemon (Citrus limonum); lavender (Lavandula angustifolium); lavandin (Lavandula hybrida grosso); kanuka (Kunzea ericoides); juniper (Juniperus cummunis); jasmine (Jasminum officinale); jasmine (Jasminum sambac); helichrysum (Helichrysum italicum); grapefruit (Citrus xparadisi); grapefruit (Citrus paradisi); ginger (Zingiber officinalis); geranium (Pelargonium graveolens); geranium (Pelargonium graveolens, 'Herit); gardenia (Gardenia jasminoides); galbanum (Ferula galbaniflua); frankincense (Boswellia carterii); frangipani (Plumeria alba); fir needle white (Abies alba); fir needle Siberia (Abies siberica); fir needle Canada (Abies balsamea); fennel (Foeniculum vulgare); Eucalyptus smithii. Eucalyptus radiata, Eucalyptus globulus, Eucalyptus citriodora, Eucalyptus Blue Mallee (Eucalyptus polybractea); elemi (Canarium luzonicum); dill (Anethum graveolens); cypress (Cupressus sempervirens); cumin (Cuminum cyminum); coriander (Coriandum sativum); cocoa (Theobroma cacao); clove (Eugenia caryophylatta); clary sage (Salvia sclarea); cistus (Labdanum) (Cistus ladaniferus L.); cinnamon (Cinnamomum zeylanicum); chamomile (Anthemis nobilis); chamomile (Matricaria chamomilla); celery seed (Apium graveolins); cedarwood (Thuja plicata); cedarwood (Juniperus virginiana); cedarwood atlas (Cedrus atlantica); carrot seed (Daucus carota); cardamon (Elettaria cardamomum); caraway seed (Carum carvi); cajeput (Melaleuca cajuputi); cade (Juniperus oxycedrus); birch (Betula alba); birch (Betula lenta); bergamot (Citrus bergamia); bay laurel (Laurus nobilis); basil (Ocimum basilicum); basil (Ocimum sanctum); basil (Ocimum basilicum); balsam poplar (Populus balsamifera); balsam Peru (Myroxylon balsamum); or angelica (Angelica archangelica L.).

In some embodiments, the methods described herein include extraction of yerba mate. As used herein, the term “yerba mate” has its ordinary meaning as understood in light of the specification, and refers to the medicinal plant, Ilex paraguariensis.

Yerba Mate, dried leaves and stemlets of the perennial tree Ilex paraguariensis St Hilaire (Luxner, 1995) are commonly used to prepare infusions widely consumed in South America. The tea like infusion was claimed by its depurative, diuretic and stimulant properties. It was reported that yerba mate helps to increase the antioxidant defense of the organism against free radicals. Yerba mate also displays an antiglycation action, which contributes to avoid diabetic chronic complications. Consumption of yerba mate infusions is important for nutritional purposes since yerba mate is a rich source of minerals, such as K, Mg, and particularly the trace element Mn. Besides minerals, yerba mate infusions contain xanthines such as caffeine, theobromine and theophylline, saponins and caffeoylquinic acids, a family of mono- and di-acyl quinic acids.

Typically, a 150 mL cup of yerba mate tea contains 10-78 mg of caffeine, (Heck & Mejia, 2007), and the level can vary depending on the amount of tea, the volume of boiled water used, the brewing temperature and the length of brewing time. In the human body, the low level of caffeine achieved by drinking such beverages mostly works as a mild central nervous stimulant. Caffeine is a natural chemical with stimulant effects. It is found in coffee, tea, cola, cocoa, guarana, yerba mate, and over 60 other products (Heckman et al., 2010). Studies have linked caffeine consumption with various human health benefits such as enhancement of cognitive functioning (Foskett et al., 2009; Lorist & Tops, 2003), improvement of neuromuscular coordination (Glade, 2010; Samoggia & Rezzaghi, 2021; San Juan et al., 2019), elevation of mood (Herz, 1999; Lieberman et al., 1987), relief of anxiety (GREDEN, 1974; Smith, 1988), and stimulation of the central nervous system and the cardiac muscle (Bolton & Null, 1981; Davis et al., 2003). Therefore, caffeine has been used as an additive for soft drinks and energy drinks. Caffeine has also been added to pharmaceuticals to improve analgesic effects (Derry et al., 2014; Ward et al., 1991; W.-Y. Zhang, 2001).

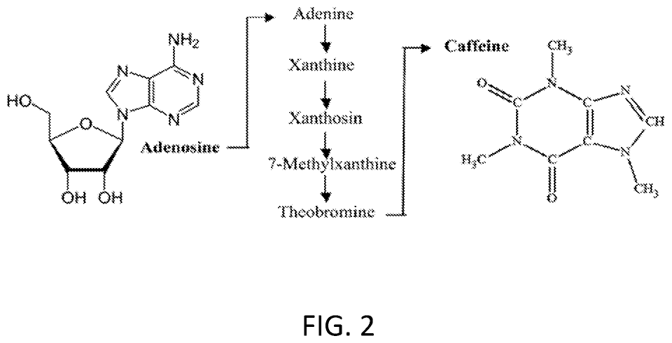

In yerba mate leaves, caffeine is the main xanthine (2-5% dry weight) with theophylline and theobromine only accounting for 0.2-0.4% and 0.02% of dry weight, respectively. Caffeine is synthesized in the tea leaves from adenosine (see FIG. 2 illustrating the biosynthesis of caffeine in tea), which is a major product of RNA metabolism throughout the life of the tea plant (Ashihara & Suzuki, 2004).

However, even at low quantities, caffeine may cause irritation of the gastrointestinal tract, fatigue and sleep deprivation in some people (Marriott, 1994; Nawrot et al., 2003; Ranheim & Halvorsen, 2005; Wikoff et al., 2017; Wolde, 2014). In addition, when taken in large quantities (>5 g), caffeine can be severely toxic with grave effects including arrhythmia, tachycardia, vomiting and convulsions and it can even cause coma and death (Andrade et al., 2018; Nojima et al., 2019). Caffeine-containing beverage consumption has been reported to be associated with reduced bone mass and increased fracture risk in some, but not most, observational studies.

Therefore, there is also an increasing demand for decaffeinated versions of the common beverages such as tea and coffee, from those who would like to consume these beverages without the potential harmful effects of caffeine. In response to this demand, decaffeinated versions of coffee have been widely available (Ramalakshmi & Raghavan, 1999; Ogita et al., 2003; Reis et al., 2010). Similarly, there is a demand for decaffeinated versions of tea and therefore, there is an increasing interest in the methods used for the decaffeination of these teas (Banerjee & Chatterjee, 2015; Dong et al., 2011; Liang et al., 2007; Park et al., 2007; Ye et al., 2009). However, because of an increasing environmental awareness among consumers, methods that are environmentally friendly and sustainable are also more likely to be the most acceptable techniques for the decaffeination of tea (Bermejo et al., 2013; Choung et al., 2014; Pietsch, 2017).

Numerous methods have been used to remove caffeine and to produce decaffeinated tea. Organic solvents such as chloroform, methylene chloride, isopropanol, and ethyl acetate have been effectively used (Choung et al., 2014; Kanda et al., 2013; Villanueva-Bermejo et al., 2017). However, consumers have increasing health concerns about the use of organic solvents in food processing and therefore, alternatives have been studied. Supercritical fluid extraction with carbon dioxide has shown potential for the decaffeination of green tea (De Marco et al., 2017; do Espirito Santo et al., 2021; Lack & Seidlitz, 1993; Lee et al., 2007; Park et al., 2007, 2012; Peker et al., 1992; Sun et al., 2010; Zabot, 2020; Zosel, 1978; Lack & Seidlitz, 1993). The process is fast, leaves no toxic residue, produces less degradation of the catechins, and lower amounts of the flavor is lost. However, it is costly to set up on an industrial scale. Microorganisms have been found that can reduce caffeine in tea extracts (Dash & Gummadi, 2006; Gokulakrishnan et al., 2005; Mazzafera, 2002; Nanjundaiah et al., 2016; Vuong & Roach, 2014), but this biotechnological method requires strictly controlled conditions, which increase the difficulties and the costs. The use of water as the only solvent used in the decaffeination process has also been studied because it is without the inherent health risks of organic solvents (Katz, 1987; Liang et al., 2007). Simply blanching the tea in boiling water for 3 to 10 minutes has been found to work quite well and the method is relatively inexpensive and relatively easy to scale up to industrial production. However, some of the other important bioactive components are also partially lost during the blanching process. Decaffeination using absorbents, including activated carbon, has also shown promise (Abebe et al., 2022; Lu et al., 2010; Oni et al., 2022; Roy, 1994; Saloko et al., 2020; Shiono et al., 2017; Quintero-Jaramillo et al., 2021). The adsorption methods include preparative paper chromatography, preparative column chromatography on silica gel and polyamide, medium pressure liquid chromatography, and/or Sephadex LH-20(size) column chromatography. These methods are inefficient, need longer time and more solvent consumption, and result in the lower recoveries of the products.

Greater attention has been paid to macroporous resins due to their excellent performance in the separation and purification of pharmaceutical and natural products (Du et al., 2012; Jin et al., 2015; A. Li et al., 2016; C. Li et al., 2011; H. Li et al., 2019; J. Li & Chase, 2010; Lin et al., 2012; Y. Liu et al., 2011; Z. Liu et al., 2013; Ma et al., 2009; Xiong et al., 2014; L. Zhang et al., 2018; Q.-W. Zhang et al., 2018; W. Zhang et al., 2016; Y. Zhang et al., 2008). As used herein, the term “macroporous adsorption resin” (MAR) has its ordinary meaning as understood in light of the specification, and refers to synthetic porous crosslinked polymer beads used to separate and purify pharmaceutical and natural products. MARs may have different physical properties such as surface area, average pore diameter, particle diameter, polarity, and attached functional groups.

The principle of adsorption is based on electrostatic forces, hydrogen bonding interactions, and size sieving action between macro-porous resins and different molecules in the solution. They have the advantages of good stability, high adsorption capacity and selectivity, fast adsorption and desorption, low operational expense, and easy regeneration of the adsorbent. Thus, they are used in very different application fields (for example, pharmaceutical, chemical, and food industries).

In some embodiments provided herein, MARs are used in extraction processes. Physical properties of six exemplary macroporous resins are presented in Table 1.

TABLE 1
Physical properties of six macroporous resins

| Trade | Surface area | Average pore | Particle | |
|---|---|---|---|---|
| name | (m²/g) | diameter (nm) | diameter (nm) | Polarity |
| HDP200 | 650-700 | 8.5-9.0 | 0.3-1.25 | Non-polar |
| HDP722 | 485-530 | 13.0-14.0 | 0.3-1.25 | Weak-polar |
| HDP400 | 500-550 | 7.5-8.0 | 0.3-1.25 | Middle polar |
| HDP750 | 650-700 | 8.5-9.0 | 0.3-1.25 | Middle polar |
| HDP600 | 550-600 | 8 | 0.3-1.25 | polar |
| HDP826 | 500-600 | 9-10 | 0.3-1.25 | H-bond |

In some embodiments, NaDES are used in extraction processes. As used herein, the term “natural deep eutectic solvent” (NaDES) has its ordinary meaning as understood in light of the specification, and refers to a sugar, amino acid, or organic acid that is typically solid at room temperature, but when combined at a particular molar fraction, present a high melting point depression, becoming a liquid at room temperature.

The discovery of the deep eutectic solvents (DES) is a major breakthrough in the world of green chemistry. Deep eutectic solvents are frequently defined as binary or ternary mixtures of compounds that are able to associate mainly via hydrogen bonds. Combining these compounds at a certain molar ratio results in a eutectic mixture. These solvents are composed of two or more inexpensive nontoxic components, one of them with the capacity to be a hydrogen bond acceptor, while the other possesses the properties of a hydrogen bond donor. Due to the formation of intramolecular hydrogen bonds and Van der Waals interactions, these solvents have much lower melting point than that of its individual components. An example of a deep eutectic is the mixture of hydroxyethyltrimethyl ammonium (choline chloride) (melting point Tₘ=302° C.) with urea (Tₘ=133° C.), which results in a eutectic of very low melting point (Tₘ=12° C.). What is interesting about these solvents is that they are not only liquid at ambient temperature but also tunable and highly solubilizing (Abbott et al., 2004).

Natural deep eutectic solvents (NaDESs) are a particular class of DES, prepared from biomolecules, such as choline chloride and betaine as the organic salt, and urea, organic acids, amino acids, or sugars as the hydrogen bond donor (Dai, 2006, 2007). This category covers the DES that are made of primary metabolites such as organic acids, amino acids, sugars, polyols, and choline derivatives (Dai et al., 2013). In addition, water may also be part of a NaDES composition. Remarkably, NaDESs are ubiquitously present in living organisms both in the intracellular and extracellular media where they may play a role in the synthesis and solubilization of poorly soluble metabolites such as flavonoids, in enzymatic reactivity, and also in drought tolerance. In this paradigm, NaDESs constitute a third type of natural liquid, separated from water and lipids (Choi et al., 2011). A series of NaDESs have been formed from abundant biomolecules, including combinations of choline chloride with citric, malic, maleic, and ascorbic acids; proline with citric acid; malic acid with glucose; and mixtures of sugars (for example, fructose:glucose, fructose:sucrose, glucose:sucrose). NaDESs are regarded as an environmentally friendly alternative solvent for the extraction of biomolecules (Liu et al., 2018).

NaDESs exhibit favorable characteristics as solvents, such as low vapor pressure, nonflammability, low or negligible toxicity, environmentally friendly, ease of preparation, and low cost. As NaDES species exhibit a superior solubilizing ability for natural products, this provides a special advantage for NaDES as extraction media. The most prominent functional groups of NaDES constituents are carboxylic acids, hydroxyl groups, and carbonyl groups. In a NaDES matrix, these groups can form a hydrogen-bonding network via intermolecular interactions that modify their physicochemical environment. Generally, the greater the intermolecular attractions, the larger the polarity. Thus, polarity is generally a solubilization property. The unique intermolecular interactions or arrangements of a NaDES matrix generate its special solubilizing and stabilizing properties. For example, while proline is only sparingly soluble in dimethylsulfoxide (DMSO), a proline-based multiple component NaDES can be fully miscible with the same organic solvent.

NaDESs possess biological activity, and can be designed with specific biologically activity. For example, if a solvent with antioxidative or/and antitumor activity is required, NaDES may be prepared with compounds that possess the desired biological activity. Previously, it was demonstrated that NaDES used for extraction purposes could enhance the antioxidative activities of obtained plant extracts, which could be explained by the reactive oxygen species scavenging activities of the NaDES itself or NaDES forming compounds. The antioxidative activity of these NaDESs was not unexpected because the forming compounds (malic acid, citric acid, proline, and betaine) also possess antioxidative activity. Furthermore, since the components of NaDES are abundant in the nature and are part of our daily diet, as well as a food supplement already present on the market (e.g., choline, citric acid, betaine, amino acids, etc.) it is expected that extract obtained by NaDES may be directly used in food, pharmaceutical, cosmetical and agrochemical products without the need for expensive downstream purification steps. NaDES also enhance the biological activity of phenolic acids (Faggian et al., 2016), and may be used directly in food, cosmetic, and pharmaceutical formulations.

NaDESs are ubiquitously present in living organisms both in the intracellular and extracellular media, where they play a role in synthesis and solubilization of poorly soluble metabolites, such as flavonoids. NaDESs also play a role in enzymatic reactivity and in drought tolerance. NaDESs exhibit numerous favorable characteristics, including low vapor pressure, non-flammability, low or negligible toxicity, no negative environmental impact, low cost, and ease of use. Further, the number of structural combinations of NaDESs is enormous, resulting in the ability to optimally design a combination of NaDESs for each specific application. NaDESs are biocompatible, and enhance biological activity. Thus, it is possible to optimally design compositions with NaDESs having a specific biological activity. Extracts obtained by NaDESs and having NaDESs present in the compositions may be used in food, nutraceutical, pharmaceutical, cosmetic, agrochemical, and industrial applications.

The extraction solvent is also a crucial factor in extraction efficiency. While a variety of extraction techniques have been studied, a limited number of studies have reported optimal extraction solvents for Yerba mate extraction (da Silveira et al., 2017; Júnior et al., 2019; Linares et al., 2010). A search for eco-friendly and safe solvents that could replace toxic, organic solvents without compromising efficiency has not been performed. “Classical” solvents often have the disadvantages of high vapor pressures, which might cause hazards including, e.g., low flash points, high flammability, toxicity, pollution corrosion to equipment, and high efforts of wastewater treatment. Thus “green” solvents are gaining more and more attention as public awareness of environmental issues increases and more tight environmental regulations are being implemented in increasingly more countries. The search for such green solvents led to the discovery of natural deep eutectic solvents (NaDESs). NaDESs can be synthesized by simply mixing two compounds (named hydrogen bond donor (HBD) and hydrogen bond acceptor (HBA)) together within a certain molar ratio. These two components must be capable of forming a eutectic mixture. NaDESs, usually a mixture of a salt and a hydrogen bond donor, exhibit a significantly lower melting point than that of each individual component. The interaction between the hydrogen bond donor molecule and the ion from the salt molecule, with additional effects such as cation molecular symmetry, causes the melting point depression in such mixtures (Choi & Verpoorte, 2019; Dai et al., 2013; Plotka-Wasylka et al., 2020; Vanda et al., 2018). NaDESs promise many advantages, such as easy to prepare, high purity, low toxicity, easily biodegradable, low melting points, high thermal stability, low volatility, non-flammability, and high air stability. Their ease of synthesis and wide availability from relatively inexpensive components also makes them more preferable compared to traditional organic solvents and ionic liquids for many applications (Choi & Verpoorte, 2019; Dai et al., 2013; Plotka-Wasylka et al., 2020; Vanda et al., 2018). Despite the fact that NaDESs are being vigorously tested to replace “conventional” solvents, their recovery and recycling as one of the main challenges to attain a cost-efficient as well as environmentally sound industrial application has only been studied very seldom. Without an effective recovery, most likely most of the options using NaDESs will never enter into commercial markets.

A hydrogen bond donor and acceptor may be selected based on the properties of the target compounds. For example, yerba mate extracts contain phenolic acids and alkaloids. Phenolic acids are highly polar compounds that a better solubilized in water than in non-polar solvents, whose chemical form and stability depend on pH value. It was reported that chlorogenic acids are stable to acid pH (Friedman & Jiirgens, 2000).

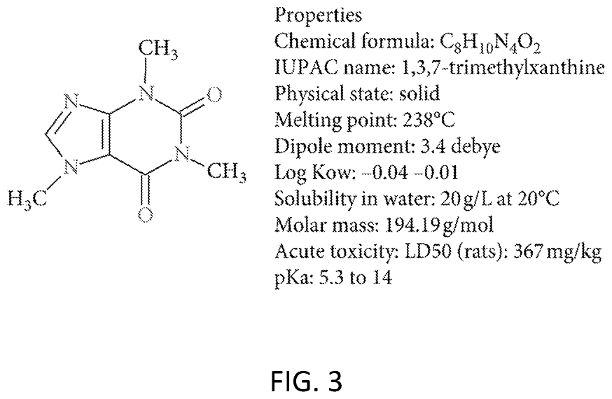

FIG. 3 presents an illustration of the chemical structure of caffeine and lists some of caffeine's properties (Quintero-Jaramillo et al., 2021). According to the information, the dipole moment of caffeine is high, increasing with the polarity of the medium that holds it. It means that the positive charge of nitrogen in caffeine electrostatically interacts with any negatively polarized functional group. The pKa and pH affect the chemical behavior, and a protonated form of caffeine will be produced when pH<pKa.

Shafie et al., 2019 reported the synthesize deep eutectic solvents using choline chloride and citric acid monohydrate at different molar ratios (for example, NaDES 3:1, 2:1, 1:1, 1:2, and 1:3) and their physicochemical characterization. They reported the NaDES was stable as a clear and viscous liquid over the course of time by POM imaging. Based on FTIR spectrum, they concluded that the formation of hydrogen bonds interactions occurred. During the formation of the NaDES, the hydroxyl groups (OH) on citric acid monohydrate as attracted to chlorine anion (Cr) in choline chloride, hence producing an OH—Cl bond. The interactions were dissimilar with the variations of molar ratios of both choline chloride and citric acid monohydrate. This was due to the fact that the presence of quaternary ammonium salt and hydrogen bond donor forms the interaction of hydrogen bonds (in particular with the hydroxyl functional groups), and thus changed the strength of the OH bond. Their results were summarized in Table 2.

TABLE 2
Physicochemical properties of NaDES by choline chloride
and citric acid monohydrate at different molar ratios
(DES 3:1, 2:1, 1:1, 1:2, and 1:3) (Shafie et al., 2019)

| | | Surface | Density, | Viscosity, Pa•S |
|---|---|---|---|---|
| NaDES | Tm (° C.) | tension, mN/m | g/ml | at 25° C. |
| ChCl:Ca 3:1 | 189.97 | 37.72 | 2.64 | 35.79 |
| ChCl:Ca 2:1 | 182.31 | 40.87 | 2.69 | 131 |
| ChCl:Ca 1:1 | 159.55 | 41.04 | 2.99 | 131 |
| ChCl:Ca 1:2 | 165.04 | 41.36 | 3.05 | 1083 |
| ChCl:Ca 1:3 | 173.80 | 41.58 | 3.11 | 1742 |

The increment of choline chloride in the mole ratio of NaDES gave a higher melting temperature and higher extraction yield. This can be explained by the more chlorine ion (Cr) in choline chloride induced stronger hydrogen bond interactions within NaDES; thus, resulting more energy were required to break the interactions, and also resulting higher extraction yield. The results showed significantly effect by decreasing mole ratio of choline chloride and increasing the molar ratio of citric acid monohydrate. The extraction yields were continuously increased with increasing the composition of citric acid monohydrate except for NaDES 1:1, which exhibit the lowest extraction yield. Thus, this indicated that the eutectic solvents might not be formed at molar ratio of 1:1. Commonly, the eutectic point was found at mole ratio of 1:2 or 2:1. For examples, the research conducted by Abbott et al., 2003 and Hayyan et al., 2013 reported that the eutectic point of choline chloride-urea and choline chloride-glucose exhibits the eutectic point at a mole ratio of 1:2 and 2:1.

The viscosity of NaDES was useful to understanding the internal resistance of a fluid to a shear stress as well as the nature of intermolecular interactions with choline chloride and citric acid monohydrate. It was reported that NaDES 1:3 showed the most viscous (1742.67 Pa s) among the synthesized NaDES due to the existence of more hydroxyl groups in citric ad monohydrate. The viscosity of NaDES increased with an increasing the molar ratio of citric acid monohydrate. The presence of more citric acid monohydrate causes more hydrogen bond interactions arise within NaDES, which increasing the attractive force and decreasing the free volume of NaDES. However, for efficient extraction purpose, lower viscosity is beneficial for mass transfer and increasing extraction yield.

The surface tension was important for interpretation of the energy required to increase the surface area of liquid as well as the intermolecular force intensity between compounds in NaDES. The data in Table 2 showed that the highest surface tension was exhibited by DES 1:2 and 1:3. The surface tension increased continuously with increasing the molar ratio of citric acid monohydrate. The temperature, interaction between quaternary ammonium salt and hydrogen bond donor, alkyl chain, viscosity and molecular weight are the main factors affecting the surface tension of NaDES. The more hydrogen bonding interaction within NaDES associate the formation of cohesive forces. Hence, the cohesive forces occurred, causing the surface to produce stronger mutual attractive forces to resist external forces and arises the surface molecules to resist surface breakage. This can be explained by the increasing the molar ratio of citric acid monohydrate as a hydrogen bond donor in DES induced more hydrogen bonding interaction formed in DES as aforementioned. However, lower surface tension system is beneficial mass transfer in extraction processing, as have observed.

The type of the quaternary ammonium salt and a hydrogen bond donor as well as the molar ratio was affecting the density of NaDES. It was noted that NaDES 3:1 (2.64 g/ml) has the lowest density followed by NaDES 2:1, NaDES 1:1, NaDES 1:2 and finally NaDES 1:3 with the highest density. The density of NaDES increased dramatically from 2.64 to 3.11 g/ml when the molar ratio of citric acid monohydrate increased. The increasing the molar ratio of citric acid monohydrate contributed to a reduction in the free space between the NaDES, thus increasing the density. Furthermore, the densities of NaDES were significantly decreased with the increment of the molar ratio of choline chloride. This could be explained by the interaction of hydroxyl groups in citric acid monohydrate complexed with chlorine anion (Cl⁻) being induced with the addition of citric acid monohydrate molar ratio. It was also responsible for the size of the formed NaDES. The packing structure and density of NaDES were also affected.

Hayyan et al., 2013 et al. reported the preparation of D-Glucose-based NaDES in different mole ratios with choline chloride and their physical properties. The results revealed that the studied NaDESs have high viscosity, density, and surface tension at room temperature hence, industrially it is more recommended to heat up these types of NaDESs before processing. However, the pH of these NaDESs were almost neutral with low sensitivity to variation of temperatures that make them good candidates for chemical, environmental, and biological applications.

In conclusion, the physicochemical properties of NaDES have great impact on extraction yield. Lower viscosity, lower density, lower surface, higher mass transfer efficiency; higher extraction yields physically. The higher melting point, the great intermolecular force and hydrogen bond, the better extraction yield chemically. It seems that viscosity is the major property that influences extraction efficiency for organic acid based NaDES since an obvious relationship was found; extraction efficiency increased as solvent viscosity decreased.

Some embodiments provided herein include choline chloride with D-glucose in the mole ratio of 2:1 as the NaDES. Choline chloride with D-glucose in the mole ratio of 2:1 has the advantage of pH stability, which is extremely important for down streaming processing and the repeatability of process.

Yerba mate extracts that were prepared in NaDES can be considered ready-to-use in the food and pharmaceutical industries, without the need for demanding the expensive downstream purification steps (Ruesgas-Ramón et al., 2017). However, the final goal is to remove the caffeine, furthermore, solvents should be recycled after processing. Volatile organic solvents that are used for extraction are usually recycled via distillation. Volatile organic solvents are a major source of industrial waste (Turnbull et al., 2004). On the other hand, NaDESs have very low vapor pressure (Lomba et al., 2019; Özel & Elibol, 2021), making them very difficult to evaporate for the easy isolation of target compounds, which may be a problem for industrial applications. Current literature suggests several possibilities for target-compounds recovery and NaDES recycling. These include liquid-liquid extraction using another solvent, solid-liquid extraction, and the addition of anti-solvents (Gullón et al., 2020; Huang et al., 2017; Vanda et al., 2018).

### Methods of Extracting a Bioactive Compound from a Plant Material

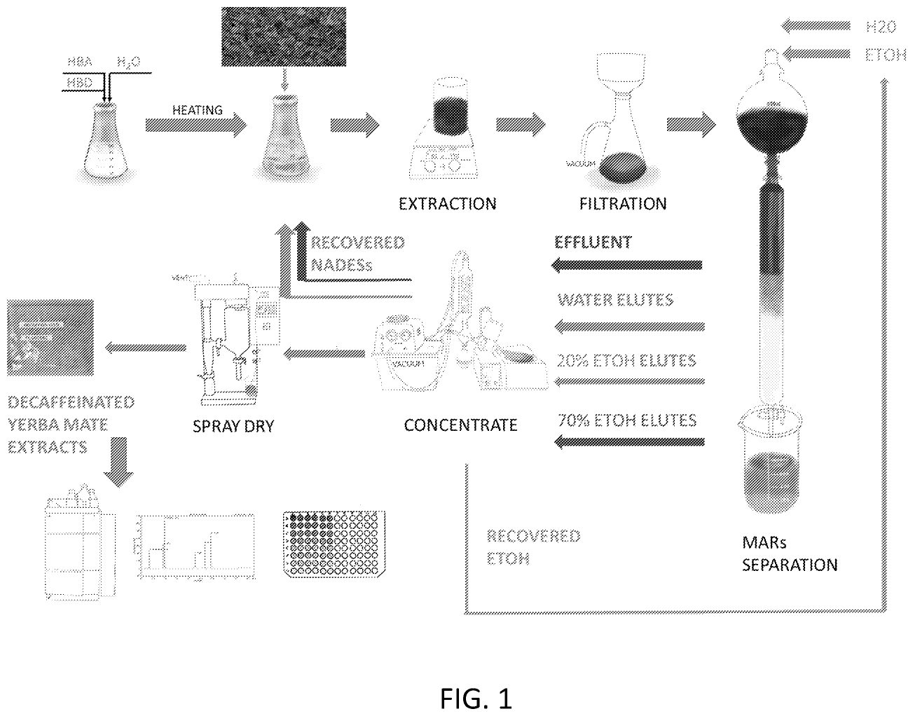

Some embodiments provided herein relate to methods of extracting a bioactive compound from a plant material. FIG. 1 illustrates a schematic flowchart representation of an exemplary process for making decaffeinated yerba mate extracts and for recovering NaDES.

In some embodiments, the methods include obtaining a plant material. In some embodiments, the methods further include mixing the plant material with a natural deep eutectic solvent (NaDES). In some embodiments, the methods further include obtaining a crude extract solution comprising extracts of the plant material, the bioactive compound, and NaDES. In some embodiments, the methods further include loading the crude extract solution onto a macroporous resin. In some embodiments, the methods further include recovering plant extract from the macroporous resin, wherein the bioactive compound and NaDES have been removed from the plant extract. In some embodiments, the bioactive compound is caffeine. In some embodiments, the plant material is obtained from yerba mate.

In some embodiments, the NaDES includes a metal salt and an organic salt, a metal salt hydrate and an organic salt, an organic salt and a hydrogen bond donor, or a metal salt hydrate and a hydrogen bond donor. In some embodiments, the NaDES include acetamide, acetic acid, 1,4-butanediol, choline acetate, choline chloride, choline fluoride, choline nitrate, citric acid monohydrate, ethylene glycol, fructose, glucose, glycerol, glycine, imidazole, lactic acid, lactose, malonic acid, maltose, mannitol, 1-methylurea, oxalic acid, phenylacetic acid, propionic acid, raffinose, resorcinol, sorbitol, sucrose, 2,2,2-trifluoroacetamide, urea, and xylitol, or any combination thereof. In some embodiments, the NaDES includes choline chloride (ChCl) and glucose (GLU). In some embodiments, the glucose is D-glucose. In some embodiments, the NaDES includes citric acid monohydrate.

The combination of components may be present in a ratio ranging from about 10:1 to about 1:10, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, or any ratio within a range defined by any two of the aforementioned values. In some embodiments, the choline chloride and glucose are present in a ratio of about 2:1. In some embodiments, the choline chloride and glucose are present in a ratio of about 3:1. In some embodiments, the choline chloride and glucose are present in a ratio of about 1:1. In some embodiments, the choline chloride and glucose are present in a ratio of about 1:2. In some embodiments, the choline chloride and glucose are present in a ratio of about 1:3.

In some embodiments, the NaDES includes water present in an amount ranging from 10%-50%, for example, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% water by weight, or in an amount within a range defined by any two of the aforementioned values. In some embodiments, the NaDES includes water present in an amount of about 30% by weight.

In some embodiments, the methods also include diluting the crude extract with water before applying the crude extract to the macroporous resin. In some embodiments, the dilution is about a 10× dilution. In some embodiments, the dilution is about a 9× dilution. In some embodiments, the dilution is about an 8× dilution. In some embodiments, the dilution is about a 7× dilution. In some embodiments, the dilution is about a 6× dilution. In some embodiments, the dilution is about a 5× dilution. In some embodiments, the dilution is about a 4× dilution. In some embodiments, the dilution is about a 3× dilution. In some embodiments, the dilution is about a 2× dilution. In some embodiments, the dilution is about a 11× dilution. In some embodiments, the dilution is about a 12× dilution. In some embodiments, the dilution is about a 13× dilution. In some embodiments, the dilution is about a 14× dilution. In some embodiments, the dilution is about a 15× dilution. In some embodiments, the dilution is about a 16× dilution. In some embodiments, the dilution is about a 17× dilution. In some embodiments, the dilution is about an 18× dilution. In some embodiments, the dilution is about a 19× dilution. In some embodiments, the dilution is about a 20× dilution. In some embodiments, the dilution is an amount within a range defined by any two of the aforementioned values.

In some embodiments, the recovered plant extract is enriched for caffeoylquinic acids. In some embodiments, the recovered plant extract contains less than 0.05% caffeine by weight, for example, 0.04%, 0.03%, 0.02%, or 0.01% caffeine by weight. In some embodiments, the recovered plant extract contains less than 0.01% caffeine by weight.

In some embodiments, the macroporous resin includes a non-polar macroporous resin. In some embodiments, the macroporous resin includes a polar macroporous resin. In some embodiments, the macroporous resin includes a weakly polar macroporous resin. In some embodiments, the macroporous resin includes a middle polar macroporous resin. In some embodiments, the macroporous resin includes an H-bond macroporous resin. In some embodiments, the macroporous resin is or includes HDP200. In some embodiments, the macroporous resin is or includes HDP722. In some embodiments, the macroporous resin is or includes HDP400. In some embodiments, the macroporous resin is or includes HDP750. In some embodiments, the macroporous resin is or includes HDP600. In some embodiments, the macroporous resin is or includes HDP826. In some embodiments, the macroporous resin is in a column.

In some embodiments, recovering the plant extract includes eluting the plant extract from the macroporous resin. In some embodiments, the plant extract is eluted with a solution with ethanol in an amount ranging from 10% to 30%, for example about 10%, 11%, 12%, 13%, 14%, 15%, 16%, 17% 18%, 19%, 20%, 21%, 22%, 23%, 24%, 25%, 26%, 27%, 28%, 29%, or 30% ethanol by volume, or in an amount within a range defined by any two of the aforementioned values. In some embodiments, the solution includes about 20% ethanol by volume.

In some embodiments, the methods also include recovering NaDES from the macroporous resin. In some embodiments, recovering NaDES from the macroporous resin includes washing the macroporous resin with water, capturing effluent and water, and drying the captured effluent and water to recover the NaDES.

Accordingly, some embodiments are set forth in the following enumerated alternatives.
1. A method of extracting a bioactive compound from a plant material, the method comprising: obtaining a plant material; mixing the plant material with a natural deep eutectic solvent (NaDES); obtaining a crude extract solution comprising extracts of the plant material, the bioactive compound, and NaDES; loading the crude extract solution onto a macroporous resin; and recovering plant extract from the macroporous resin, wherein the bioactive compound and NaDES have been removed from the plant extract.
2. The method of alternative 1, wherein the bioactive compound is caffeine.
3. The method of any one of alternatives 1-2, wherein the plant material is obtained from yerba mate.
4. The method of any one of alternatives 1-3, wherein the NaDES comprises choline chloride (ChCl) and glucose (GLU).
5. The method of alternative 4, wherein the choline chloride and glucose are present in a ratio of about 2:1.
6. The method of any ones of alternatives 1-5, wherein the NaDES comprises water present in an amount ranging from 10%-50% by weight.
7. The method of any one of alternatives 1-6, wherein the NaDES comprises water present in an amount of about 30% by weight.
8. The method of any one of alternatives 1-7, further comprising diluting the crude extract with water before applying the crude extract to the macroporous resin.
9. The method of alternative 8, wherein the dilution is about a 10× dilution.
10. The method of any one of alternatives 1-9, wherein the recovered plant extract is enriched for caffeoylquinic acids.
11. The method of any one of alternatives 1-10, wherein the recovered plant extract contains less than 0.05% caffeine by weight.
12. The method of any one of alternatives 1-11, wherein the recovered plant extract contains less than 0.01% caffeine by weight.
13. The method of any one of alternatives 1-12, wherein the macroporous resin comprises a non-polar macroporous resin.
14. The method of any one of alternatives 1-13, wherein the macroporous resin comprises HDP200.
15. The method of any one of alternatives 1-14, wherein the macroporous resin is in a column.
16. The method of any one of alternatives 1-15, wherein recovering the plant extract comprises eluting the plant extract from the macroporous resin.
17. The method of alternative 16, wherein the plant extract is eluted with a solution comprising ethanol in an amount ranging from 10% to 30% by volume.
18. The method of alternative 17, wherein the solution comprises about 20% ethanol by volume.
19. The method of any one of alternatives 1-18, further comprising recovering NaDES from the macroporous resin.
20. The method of alternative 19, wherein recovering NaDES from the macroporous resin comprises washing the macroporous resin with water, capturing effluent and water, and drying the captured effluent and water to recover the NaDES.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Materials and Methods

Materials and Chemicals: Yerba mate leaves were sourced from Triunfo and used as received. Citric acid, glucose and choline chloride were purchased from TCI chemicals. HPLC grade water, methanol, acetonitrile, LC grade formic acid were purchased from Fisher Chemicals. Reference standards chlorogenic acid, caffeine, theobromine, rutin, 3,4-dicaffeoylquinic acid, 3,5-dicaffeolyquinic acid, 4,5-dicaffeoylquinic acid were purchased from Sigma chemicals. 2,2-diphenyl-1-picrylhydrazyl (DPPH), a product of Caymon Chemicals, was purchased from i-DNA Biotechnology, SG.

Macroporous Resins: Six macroporous resins (HDP200, HDP400, HDP600, HDP722, HDP750 and HDP826) were purchased from Bonchem Co. Ltd (Guanghou, China). Their physical properties were listed in Table 1. Briefly, the resins were pre-treated by soaking in ethanol for 24 hr. They were then rewashed with fresh ethanol adequately, distilled until no oligomers, porogenic agents and other ethanol-dissolved substances were left, and dried in a drying oven at 70° C. Prior to use, the resins were soaked in ethanol and washed thoroughly with distilled water.

NaDES Preparation: NaDES was prepared by mixing choline chloride (ChCl), which acts as a hydrogen bond acceptor (HBA), with citric acid (CA), or with glucose (GLU) which serves as an HBD. Choline chloride (ChCl) was dried in a vacuum oven at 60° C. for 24 hr before use. ChCl and citric acid (CA) in the molar ratio of 3:1, 2:1, 1:1, 1:2, 1:3 ChCl and glucose (GLU) in the molar ratio of 2:1 with 30% water were placed in a glass beaker, and then were stirred using a magnetic stirrer (multi-channel magnetic stirrer, HS-8, Yooning, Hangzhou, China) with the speed scale set 350 rpm. The glass beaker was covered with Parafilm and heated to 50° C. for 2 hours until a homogeneous transparent colorless liquid was formed.

Preparation of NaDES Extract: The NaDES extraction process was performed using a water bath shaker (WS-10, Yooning, Hangzhou, China). The yerba mate leaves 3 g were added to a 50 ml centrifuge tube and then mixed with 30g solvents, both NaDES and other organic solvents. The extraction was performed for 3 hours at 60° C. The mixtures were centrifuged (LC-8S, Joan Lab equipment, Huzhou, China) for 8 min at 4000 rpm to separate the NaDES liquid extract from biomass. The filtrates were collected and stored in a refrigerator until future analysis.

Static adsorption/desorption properties of macroporous resins: Static adsorption/desorption tested were performed to select the proper macroporous resin for removal of caffeine and recovery of the other bioactives and NaDES. Pre-treated hydrated resins (3.0 g, dry weight) were put into 250 ml Erlenmeyer flasks, following by addition of 40 ml sample solutions. Subsequently, the flasks were sealed tightly with a stopper and continually shaken in an orbital shaker at 120 r/min for 12 hr at 25° C. The contents of bioactives in the solution before and after adsorption were analyzed by HPLC.

After adsorption equilibrium was reached, the adsorbate-laden resins were first filtrated, thoroughly washed by distilled water three times, and desorbed with 50 ml 70% (v/v) ethanol. The flasks were continually shaken at 120 rpm for 12 hr at 25° C. The contents of bioactives in the desorbed solutions were analyzed by HPLC.

The following formulas were used to calculate the adsorption capacity (Qₑ), desorption capacity (Q_{d}) and desorption ratio (D).

$$Adsorption capacity {Q}_{e} = \frac{( {C}_{0} - {C}_{e} ) Vi}{W}$$

$$Desorption capacity {Q}_{d} = \frac{{C}_{d} {V}_{d}}{W}$$

$$Desorption ratio D = \frac{{C}_{d} \times {V}_{d}}{( {C}_{0} - {C}_{e} ) {V}_{i}} \times 100 \%$$

Where Qₑ is adsorption capacity at adsorption equilibrium (mg/g, dry resin); Q_{d} is the desorption capacity after adsorption equilibrium (mg/g, dry resin); C₀ and Cₑ are the initial and adsorption equilibrium concentration in the solutions (mg/ml); C_{d} is the concentration in the desorption solutions (mg/ml); Vᵢ and V_{d} are the volumes of the initial sample and desorption solutions (ml). D is the desorption ratio (%). W is the weight of dry resin (g).

Caffeine removal, chlorogenic acids recovery and NaDES recycling: Caffeine removal, chlorogenic acids recovery and NaDES recycling was achieved via macroporous resin adsorption on a glass column (2.5 cm×24.5 cm), which was wet-packed with 80 g HDP200 (BV=120 ml). Resin was pre-treated with 2 BV of 96% ethanol and again with 3 BV of deionized water. 15 ml of the extracts prepared with ChClGlu 2:1 was diluted 10 times with 135 ml of water. 150 ml extract solution flowed through the column at flow rate of 1.14 BV/hr. ChClGlu was eluted from the column with 1.6 BV deionized water; decaffeinated, chlorogenic acids enriched yerba mate extracts was eluted from the column with 1.6 BV 20% v/v ethanol; other bioactives in yerba mate extracts was eluted from the column with 1.6 BV 70% v/v ethanol. The water fraction evaporated under vacuum and ChClGlu was recovered. 20% ethanol and 70% ethanol fractions were analyzed by HPLC and yields were calculated.

High performance liquid chromatography (HPLC) analysis: The quantitative analysis of bioactives was determined using Thermo Fisher Vanquish series HPLC system, equipped with Chromeleon software, a VC-P10-A-01 pump and a VC-D11-A-01 diode array detector (DAD). The liquid chromatographic separation was performed on a reversed-phase Accucore C18 column (150 mm×2.1 mm, particle size 2.6 μm, Thermo Fisher) at the oven temperature of 30° C. The detection wavelength was 280 nm for caffeine and theobromine and 350 nm for rutins and all the other chlorogenic acids, the flowrate was 0.4 ml/min and the injection volume was 10 μl for each run. The mobile phase consisted of 0.1% formic acid in water (A) and 0.1% formic acid in acetonitrile (B). The gradient elution of mobile phase was 5-20% B in 0-10 min, 20% B in 10-12 min, 20-5% B in 12-15 min, 5-95% B in 15-15.1 min, 95% B in 15.1-16.5 min, 95-5% B in 16.5-16.6 min and 5% B in 16.6-19 min. The tested samples and standards were dissolved with HPLC grade methanol and filtered through 0.22 μm PTFE (polytetrafluoroethylene) filters before use.

The chromatographic peaks of all bioactives were identified by their retention times. The retention times of theobromine, neo-chlorogenic acid, chlorogenic acid, caffeine, crypto-chlorogenic acid, rutin, 3,4-dicaffeoylquinic acid, 3,5-dicaffeoylquinic acid and 4,5-dicaffeoylquinic acid were 1.54 min, 2.48 min, 4.337 min, 4.673 min, 5.300 min, 9.937 min, 11.450 min, 11.737 min and 12.747 min, respectively. Each standard solution was prepared at concentration of 1 mg/ml in methanol and then mixed with the same volume to make the mixed solution at concentration of 1 mg/ml then stepwise diluted to 0.5, 0.25, 0.125, 0.0625, 0.03125 and 0.015625 mg/ml. For each standard, the calibration was done in the range of 2.23 μg/ml to 142.86 μg/ml or 2.23 ppm to 142.86 ppm. Above six experimental points were used to plot a calibration curve. The regression equations for theobromine, caffeine, chlorogenic acid, rutin, 3,4-dicaffeoylquinic acid, 3,5-dicaffeoylquinic acid and 4,5-dicaffeoylquinic acid were Y=0.0335X-0.0619 (R²=0.9978), Y=0.0969X+0.0434 (R²=0.9999), Y=0.0498X-0.0459 (R²0.9992), Y=0.0648X+0.0345 (R²=1.000), Y=0.0632X-0.0358 (R²=0.9992), Y=0.0812X+0.0332 (R²=0.9999) and Y=0.0819X-0.056 (R²=0.9997), respectively, where Y is the peak area, X is the concentration (μg/ml).

DPPH radicals scavenging capacity assay: The ability of the plant extract to scavenge DPPH free radicals was assessed by using the method described by Takao et al. (Takao et al., 1994).

The stock solution of the plant extract was prepared in methanol to achieve the concentration of 2000 μg/ml. Further, two-fold dilutions were made to obtain concentrations of 1000, 500, 250, 125, 62.5, 31.25, 15.62 μg/ml. Diluted solutions of extract (2 ml each) were mixed with 2 ml of DPPH methanol solution (80 μg/ml). After 30 min in darkness at room temperature, the absorbance was read in a spectrophotometer at 517 nm. The control samples consisted of 2 ml of methanol added to 2 ml of DPPH solution. Caffeoylquinic acid and ascorbic acid was used as a positive control. The experiment was performed in triplicate. Scavenging activity is expressed as the inhibition percentage calculated using the following equation: Scavenging activity (%)=100×[A_{control}−Aₛₐₘₚₗₑ)/A_{control})], where A_{control} is the absorbance of the control and Aₛₐₘₚₗₑ is the absorbance of the extract.

The IC₅₀ value is the effective concentration at which 50% of DPPH radicals were scavenged. It was obtained from the graph of scavenging activity (%) versus concentration of samples. Low IC₅₀ value indicates strong ability of the extract to act as DPPH scavenger.

Experimental setup for processing scale-up: 20 g of yerba mate leaves were extracted using 200 g of ChClGLU2:1 with 30 wt % of water. The extraction was done at 60° C. for 3 hours. In order to decrease viscosity and facility the following process, 80 g of water was added to make concentration at about 50%. The mixture was filtered by filtration. The filtrate was retained for the further processing. Caffeine removing, chlorogenic acids, bioactives recovery, NaDES recovery was achieved on a glass column (6 cm×33.5 cm), which was wet-packed with about 670 g HDP200 (bed volume(BV)=1 L). Resin was pre-treated with 2 BV of 95% ethanol again with 3 BV of deionized water. The extracts prepared above were future diluted to 90% of water and flowed through the column at flow rate of 1.14 BV/hr. ChClGLU was eluted from the column with 2 BV deionized water, de-caffeinated caffeoylquinic acid enriched fraction was desorbed with 2 BV 20% v/v aqueous ethanol; the caffeine-enriched fraction was desorbed with 2 BV 70% v/v aqueous ethanol. The effluent and water fraction were evaporated under vacuum and ChClGLU was recovered. EtOH fractions were analyzed by HPLC, and yields were calculated. Recycled NaDES were made up with water to 30% by weight and used again for extraction.

### Example 1

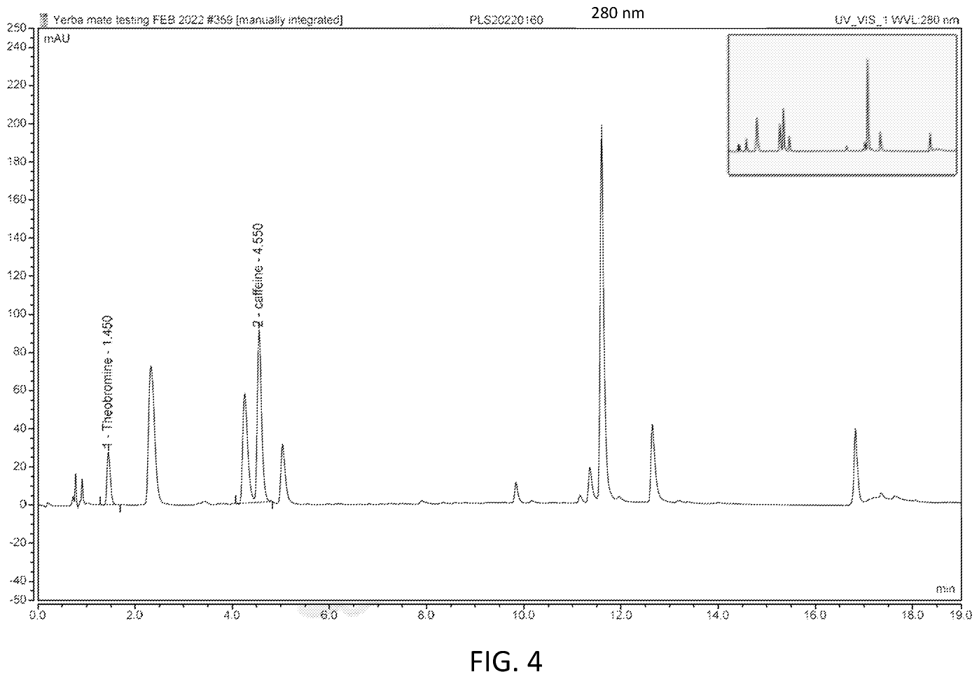

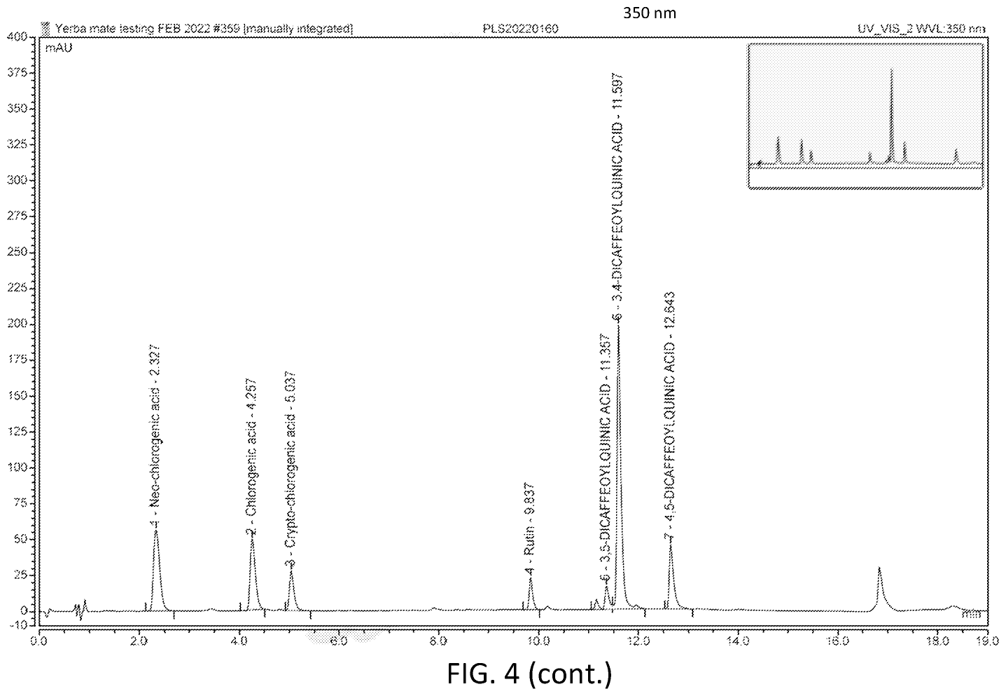

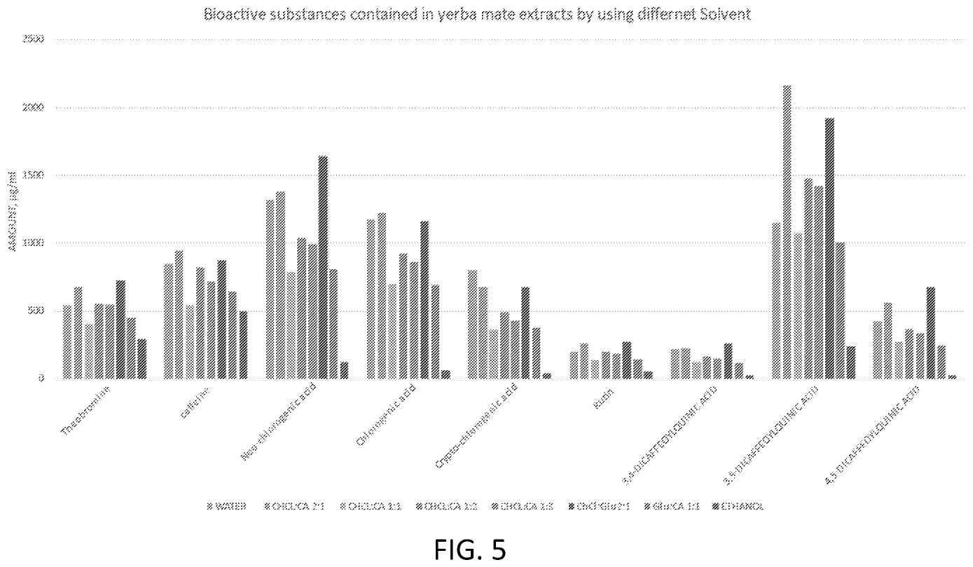

HPLC Results: HPLC testing method was successfully applied to test the phenolic acids and caffeine contained in yerba mate leaves extracts obtained under various conditions. As observed in FIG. 4, theobromine (Rt=1.45 min) and caffeine (Rt=4.55 min) were detected at 280 nm; Neo-chlorogenic acid (Rt=2.327 min), chlorogenic acid (Rt=4.257 min), Crypto-chlorogenic acid (Rt=5.037 min), rutin (Rt=9.837 min), 3,4-dicaffeoylquinic acid (Rt=11.357 min), 3.5-dicaffeoylquinic acid (Rt=11.597 min) and 4.5-dicaffeoylquinic acid (Rt=12.643 min) were detected at 350 nm. UV detector at 280 nm was used to test theobromine and caffeine and UV detector at 350 nm was used to test caffeoylquinic acid and other bioactives. All compounds were well separated. The peaks analysis table is shown in Table 3. The results illustrate that the applied chromatography method for analyzing yerba mate extract successfully separated nine biomarkers. High-resolution values indicate that the compounds peaks are separated by a baseline gap whereas 1.2 is the minimum value needed for two peak compounds to completely separate. (Ahuja, 2002).

TABLE 3
HPLC peak analysis table for bioactives in Yerba Mate extracts

| | Retention | | | | | | |
|---|---|---|---|---|---|---|---|
| | Time | Peak Area | Height | Width | Asym, | Resol, | Plate, |
| | (min) | (mAU*min) | (mAU) | (50%) | EP | EP | EP |
| Theobromine | 1.45 | 2.2 | 27.6 | 0.075 | 1.39 | 17.94 | 2050 |
| Caffeine | 4.55 | 9.5 | 91.07 | 0.094 | 1.46 | NA | 12943 |
| Neo-chlorogenic | 2.327 | 8.0 | 56.13 | 0.135 | 1.47 | 9.31 | 1642 |
| acid | | | | | | | |
| Chlorogenic acid | 4.257 | 6.0 | 49.83 | 0.109 | 1.54 | 4.49 | 8380 |
| Crypto-chlorogenic | 5.037 | 2.9 | 27.64 | 0.095 | 1.5 | 34.54 | 15455 |
| acid | | | | | | | |
| Rutin | 9.837 | 1.7 | 22.53 | 0.069 | 1.51 | 11.64 | 113828 |
| 3,4-dicaffeoylquinic | 11.357 | 1.4 | 15.53 | 0.077 | NA | 1.82 | 119445 |
| acid | | | | | | | |
| 3,5-dicaffeoylquinic | 11.597 | 17.7 | 196.81 | 0.078 | 1.67 | 7.46 | 121142 |
| acid | | | | | | | |
| 4,5-dicaffeoylquinic | 12.643 | 4.6 | 44.6 | 0.087 | 2.05 | NA | 116395 |
| acid | | | | | | | |

### Example 2

NaDESs were prepared with 30 wt % of water in order to decrease viscosity and improve mass transfer between the solid and liquid phases. Results were compared with ethanol, as it was the most suitable and green conventional organic solvent. The concentration of chlorogenic acid in ethanol extracts was only 61 μg/ml, but it was 1221 μg/ml in ChCl:CA2:1, that was 20 fold. The total caffeoylquinic acid (Total CQAs), the extraction yield increased 10 times from 528 μg/ml in ethanol to 6346 μg/ml in ChClGlu 2:1. The results showed that NaDESs are remarkably superior to organic solvents

All of the citric acid based NaDESs were polar, with polarities similar to water, and pH values were from 0.16-1.62. The results also revealed that the extraction yields obtained by NaDESs were similar to water, but change in molar ratios did affect extraction efficiency significantly. The best extraction efficiency for extraction bioactive substances in yerba mate was found by using chlorine chloride/citric acid in mole ratio of 2:1 or chlorine chloride/glucose in mole ratio of 2:1. The lowest extraction efficiency was found by using chlorine chloride/citric acid or glucose/citric acid in mole ratio of 1:1. The ability of NaDES to extract bioactive substances from the natural product matrix correlated well with its physical and chemical properties such as hydrogen bonding, polarity, acidity, and viscosity.

The content of bioactive substances in NaDES extracts are presented in Table 4 and FIG. 5. Total CQA, total caffeoylquinic acid, was calculated by combining mono-caffeoylquinic acid and di-caffeoylquinic acid.

TABLE 4
Results of the determination bioactive substances contained in yerba mate extracts in amount of ug/ml

| | | | | | Neo- | | Crypto- | | 3,4- | 3,5- | 4,5- | |
|---|---|---|---|---|---|---|---|---|---|---|---|---|
| | | | | | chloro- | Chloro- | chloro- | | dicaffe- | dicaffe- | dicaffe- | |
| Sample | | | Theo- | | genic | genic | genic | | oylquinic | oylquinic | oylquinic | Total |
| No. | Solvent | pH | bromine | Caffeine | acid | acid | acid | Rutin | acid | acid | acid | CQA |
| 1 | ChCl:Ca | 1.62 | 538 | 757 | 1124 | 975 | 537 | 217 | 197 | 1578 | 405 | 4816 |
| | 3:1 | | | | | | | | | | | |
| 2 | ChCl:Ca | 1.12 | 681 | 946 | 1379 | 1224 | 679 | 262 | 227 | 2165 | 565 | 6239 |
| | 2:1 | | | | | | | | | | | |
| 3 | ChCl:Ca | 0.52 | 407 | 542 | 785 | 699 | 361 | 141 | 127 | 1073 | 272 | 3319 |
| | 1:1 | | | | | | | | | | | |
| 4 | ChCl:Ca | 0.24 | 559 | 821 | 1043 | 924 | 491 | 197 | 168 | 1480 | 370 | 4476 |
| | 1:2 | | | | | | | | | | | |
| 5 | ChCl:Ca | 0.16 | 547 | 720 | 991 | 860 | 431 | 189 | 154 | 1425 | 338 | 4199 |
| | 1:3 | | | | | | | | | | | |
| 6 | Glu:Ca1:1 | 0.93 | 456 | 643 | 810 | 690 | 375 | 149 | 120 | 1005 | 245 | 3245 |
| 7 | ChCl:Glu | 5.53 | 728 | 877 | 1642 | 1161 | 677 | 277 | 263 | 1923 | 681 | 6346 |
| | 2:1 | | | | | | | | | | | |
| 8 | Ethanol | 5.98 | 294 | 501 | 125 | 61 | 43 | 55 | 27 | 242 | 30 | 528 |
| 9 | Water | 5.51 | 541 | 853 | 1322 | 1180 | 799 | 203 | 221 | 1153 | 425 | 5100 |

### Example 3

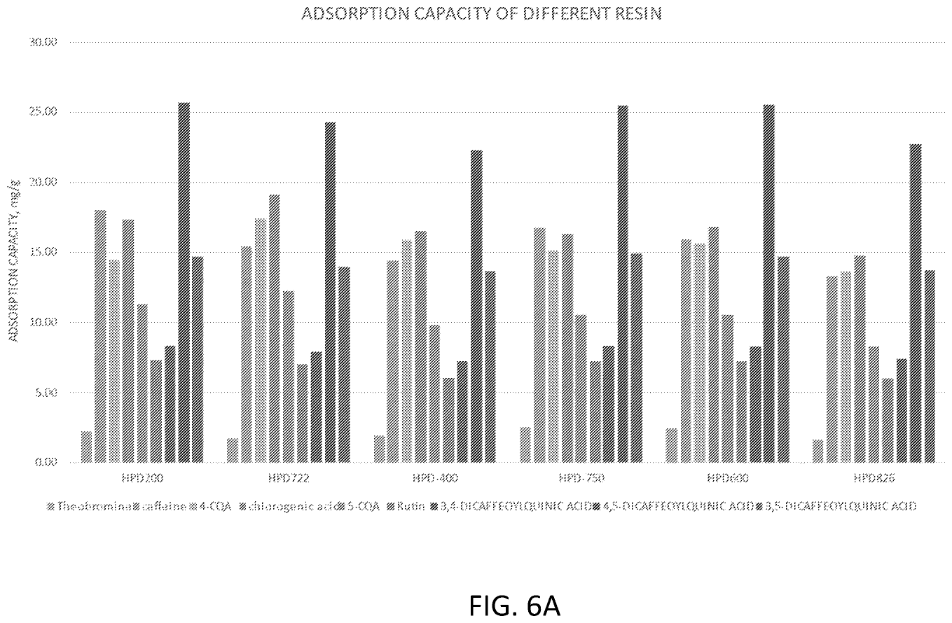

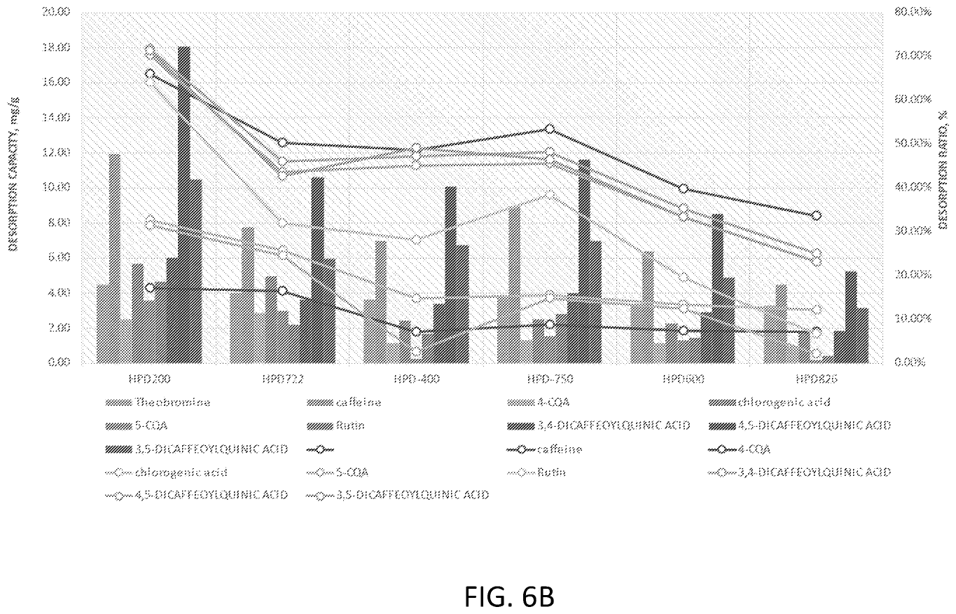

Macroporous resin selection: Commercial HDP series resins with different polarity and physical properties were selected for study. These resins were prepared by post-crosslinking chloromethylated gel-type styrene-DVB copolymer via Friedel-Crafts reaction followed by treating residual chloromethyl groups with dimethylamine. The polarity was adjusted by adding in a functional group. In addition to chemical structures and polarities, the adsorption/desorption characteristics of macroporous resins are closely related to their physical properties, such as surface area and pore diameter. Since both caffeine and caffeoylquinic acids contain non-polar benzene rings, according to the principle of similarity and intermiscibility for the resin adsorptive selectivity, the non-polar, polar or H-bond resins were selected to optimize the process. Static adsorption and desorption experiments results are shown in Table 5 and FIGS. 6A-6B.

TABLE 5
The adsorption and desorption capacities (mg/g), and desorption ratio (%)
of yerba mate bioactives on different kinds of macroporous resins at 25° C.
(A) Adsorption Capacities (mg/g) of yerba mate bioactives

| | | | Neo- | | Crypto- | | 3,4- | 4,5- | 3,5- |
|---|---|---|---|---|---|---|---|---|---|
| | | | chlorogenic | Chlorogenic | chlorogenic | | dicaffeoyl- | dicaffeoyl- | dicaffeoyl- |
| Resin | Theobromine | Caffeine | acid | acid | acid | Rutin | quinic acid | quinic acid | quinic acid |
| HDP200 | 2.24 | 18.01 | 14.47 | 17.33 | 11.30 | 7.30 | 8.37 | 25.68 | 14.69 |
| HDP722 | 1.73 | 15.41 | 17.43 | 19.12 | 12.27 | 7.01 | 7.90 | 24.29 | 13.97 |
| HDP400 | 1.92 | 14.39 | 15.85 | 16.53 | 9.82 | 6.04 | 7.25 | 22.29 | 13.69 |
| HDP750 | 2.51 | 16.73 | 15.15 | 16.36 | 10.54 | 7.25 | 8.34 | 25.47 | 14.95 |
| HDP600 | 2.48 | 15.99 | 15.69 | 16.83 | 10.60 | 7.25 | 8.29 | 25.53 | 14.68 |
| HDP826 | 1.63 | 13.28 | 13.64 | 14.80 | 8.26 | 6.03 | 7.40 | 22.75 | 13.72 |

(B) Desorption capacities (mg/g) of yerba mate bioactives

| | | | Neo- | | Crypto- | | 3,4- | 4,5- | 3,5- |
|---|---|---|---|---|---|---|---|---|---|
| | | | chlorogenic | Chlorogenic | chlorogenic | | dicaffeoyl- | dicaffeoyl- | dicaffeoyl- |
| Resin | Theobromine | Caffeine | acid | acid | acid | Rutin | quinic acid | quinic acid | quinic acid |
| HDP200 | 4.48 | 11.89 | 2.49 | 5.64 | 3.56 | 4.68 | 6.01 | 18.05 | 10.45 |
| HDP722 | 3.97 | 7.75 | 2.89 | 4.93 | 3.00 | 2.24 | 3.63 | 10.57 | 5.97 |
| HDP400 | 3.67 | 7.00 | 1.13 | 2.44 | 0.25 | 1.69 | 3.42 | 10.05 | 6.72 |
| HDP750 | 3.80 | 8.92 | 1.33 | 2.52 | 1.55 | 2.79 | 4.01 | 11.59 | 6.96 |
| HDP600 | 3.40 | 6.35 | 1.15 | 2.25 | 1.33 | 1.43 | 2.92 | 8.55 | 4.91 |
| HDP826 | 3.27 | 4.45 | 0.98 | 1.79 | 0.18 | 0.40 | 1.85 | 5.25 | 3.16 |

(C) Desorption ratio (%) of yerba mate bioactives

| | | Neo- | | Crypto- | | 3,4- | 4,5- | 3,5- |
|---|---|---|---|---|---|---|---|---|
| | | chlorogenic | Chlorogenic | chlorogenic | | dicaffeoyl- | dicaffeoyl- | dicaffeoyl- |
| Resin | Caffeine | acid | acid | acid | Rutin | quinic acid | quinic acid | quinic acid |
| HDP200 | 66.03% | 17.19% | 32.54% | 31.53% | 64.11% | 71.76% | 70.30% | 71.14% |
| HDP722 | 50.26% | 16.57% | 25.80% | 24.49% | 31.94% | 46.00% | 43.52% | 42.75% |
| HDP400 | 48.66% | 7.16% | 14.76% | 2.55% | 28.04% | 47.15% | 45.10% | 49.07% |
| HDP750 | 53.31% | 8.76% | 15.40% | 14.74% | 38.42% | 48.08% | 45.50% | 46.57% |
| HDP600 | 39.70% | 7.30% | 13.39% | 12.51% | 19.67% | 35.20% | 33.47% | 33.42% |
| HDP826 | 33.53% | 7.18% | 12.07% | 2.18% | 6.64% | 25.02% | 23.09% | 23.03% |

Table 5(A) and FIG. 6A show the absorbent capacities of six studied resins, ranging from non-polar, polar to H-bond. For caffeoylquinic acids, the adsorption capacity of HDP200, HDP400, HDP600, HDP722, HDP750 were higher than HDP826, indicating that the five resins had better adsorption selectivity for caffeoylquinic acids and rutin. The Van der Waals force and 7C-7C conjugation may contribute to their strong affinity since these reins have large surface area of 500-700 m²/g and multiple benzene rings. HDP826 showed the lowest adsorption capacity; suggest that hydrogen bond interaction was not important in a synergistic effect.

Table 5(B) and FIG. 6B shows the desorption capacity. HDP200 shows the highest desorption ratio in Table 5(C), suggesting HDP200 also had a better desorption capacity. HDP826 resin exhibited the lowest desorption ratio, which was approximately 50% lower than that of HDP200 resin, suggesting that both caffeine and chlorogenic acids were difficult to dissociate from HDP826 resin. The results show that HDP826 resin results in a significant wastage of bioactives. Therefore, HDP826 was not selected for the subsequent study.

Because caffeine was the target impurity to remove, the adsorption capacity of these resins for caffeine were examined. Among resins with a high absorption capacity for phenolic acids, non-polar HDP200 resin possessed the highest adsorption and desorption for caffeine.

Therefore, HDP200 was selected for the optimized media to recovery bioactives and remove caffeine.

### Example 4

HDP200 based column separation to remove caffeine and recover NaDES: The dynamic desorption experiment was done on a 120 ml column by using HDP200 as separation media. The results are listed in Table 6.

TABLE 6
Results of gradient elution of yerba mate bioactives on column packed with the HPD200 resin
(A) The purity

| | | | | Neo- | | Crypto- | | 3,4- | 3,5- | 4,5- | |
|---|---|---|---|---|---|---|---|---|---|---|---|
| | | | | chloro- | Chloro- | chloro- | | dicaffe- | dicaffe- | dicaffe- | |
| | | Theo- | | genic | genic | genic | | oylquinic | oylquinic | oylquinic | Total |
| Sample | Description | bromine | Caffeine | acid | acid | acid | Rutin | acid | acid | acid | CQA |
| 10 | Crude | 0.06% | 0.12% | 0.22% | 0.15% | 0.09% | 0.04% | 0.04% | 0.26% | 0.09% | 0.88% |
| | extract | | | | | | | | | | |
| 13 | 20% EtOH | 2.44% | — | 7.64% | 5.24% | 3.14% | 0.04% | 0.32% | 2.82% | 0.75% | 19.96% |
| | elutes | | | | | | | | | | |
| 14 | 70% EtOH | 2.93% | 7.61% | — | — | — | 2.38% | 2.39% | 16.33% | 5.81% | 26.91% |
| | elutes | | | | | | | | | | |

(B) The weight of bioactives

| Sample | 10 | 11 | 12 | 13 | 14 |
|---|---|---|---|---|---|
| Description | Crude extract | Effluent | Water elutes | 20% EtOH elutes | 70% EtOH elutes |
| Total Solid (g) | 11.24 | 5.40 | 3.30 | 0.20 | 0.16 |
| Step processing rate | — | 48.1% | 29.4% | 1.8% | 1.4% |
| Total processing | — | — | — | 10.1% | 7.8% |
| yield | | | | | |
| Bioactives | 5.99% | — | — | 2.26% | 2.94% |
| processing yield | | | | | |
| Theobromine (mg) | 7.28 | — | 0.05 | 4.92 | 4.59 |
| Caffeine (mg) | 13.15 | — | 0.05 | — | 11.93 |
| Neo-chlorogenic | 24.62 | — | 0.13 | 15.42 | — |
| acid (mg) | | | | | |
| Chlorogenic acid | 17.41 | — | 0.02 | 10.58 | — |
| (mg) | | | | | |
| Crypto-chlorogenic | 10.15 | — | 0.03 | 6.34 | — |
| acid (mg) | | | | | |
| Rutin (mg) | 4.16 | — | — | 0.09 | 3.73 |
| 3,4-dicaffeoylquinic | 3.95 | — | 0.01 | 0.64 | 3.75 |
| acid | | | | | |
| 3,5-dicaffeoylquinic | 28.85 | — | 0.11 | 5.68 | 25.62 |
| acid | | | | | |
| 4,5-dicaffeoylquinic | 10.22 | — | — | 1.51 | 9.11 |
| acid | | | | | |

The results indicated that in yerba mate crude extract by using NaDESs, the total extraction yield of all bioactives was 5.99%, that was what is contained in yerba mate leaves. The purity of bioactive was less than 1%, due to a large amount of NaDES solvent component present in crude extracts. Therefore, concentrating bioactive to decent concentration by removing and recovering NaDES may be an important step for practical application.

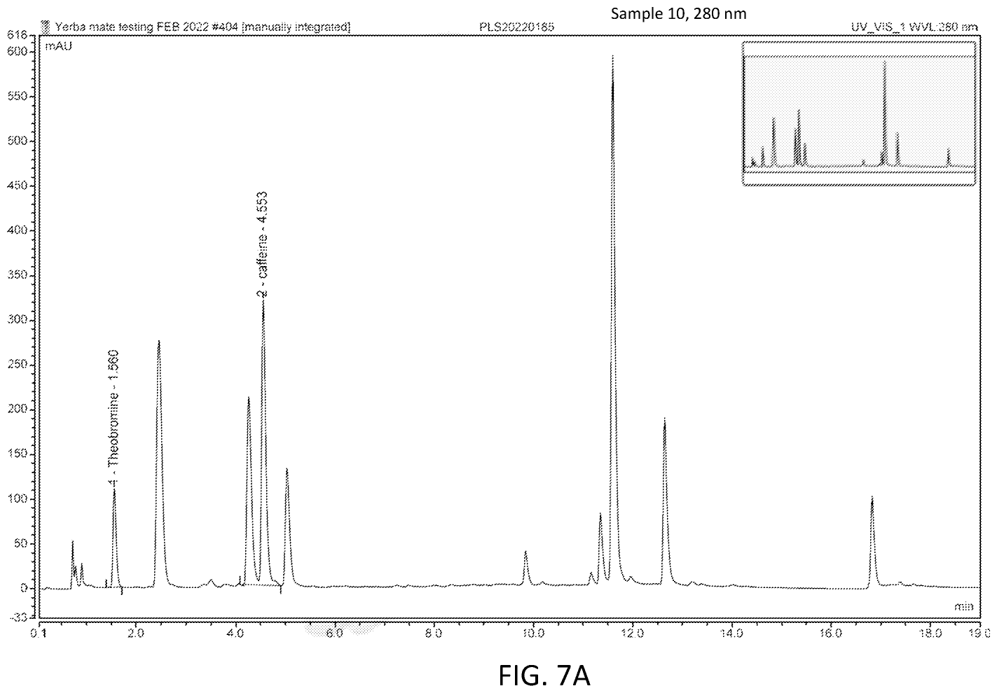

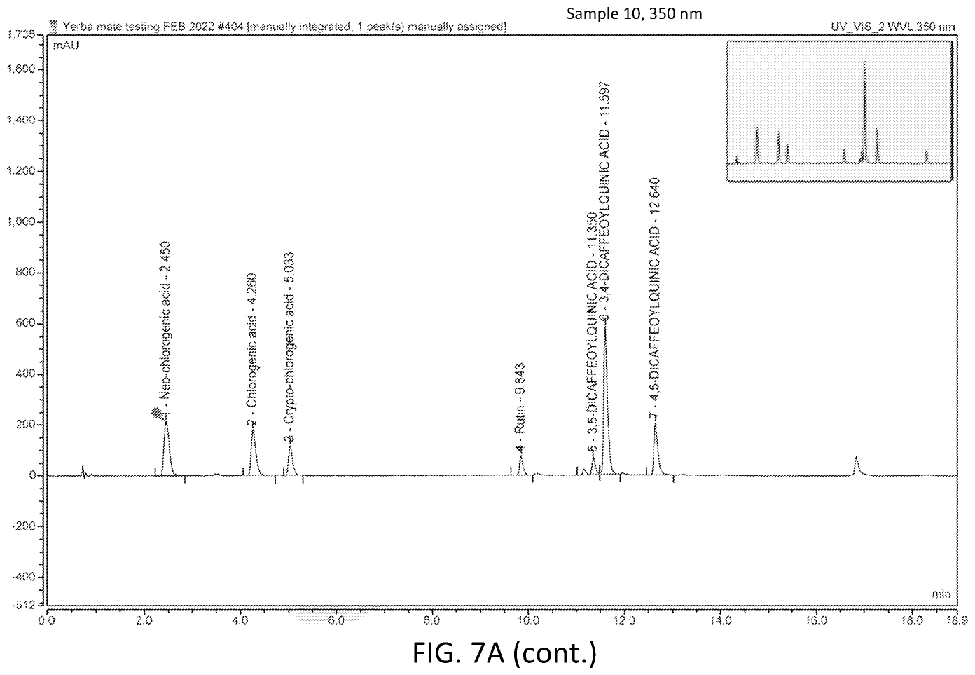

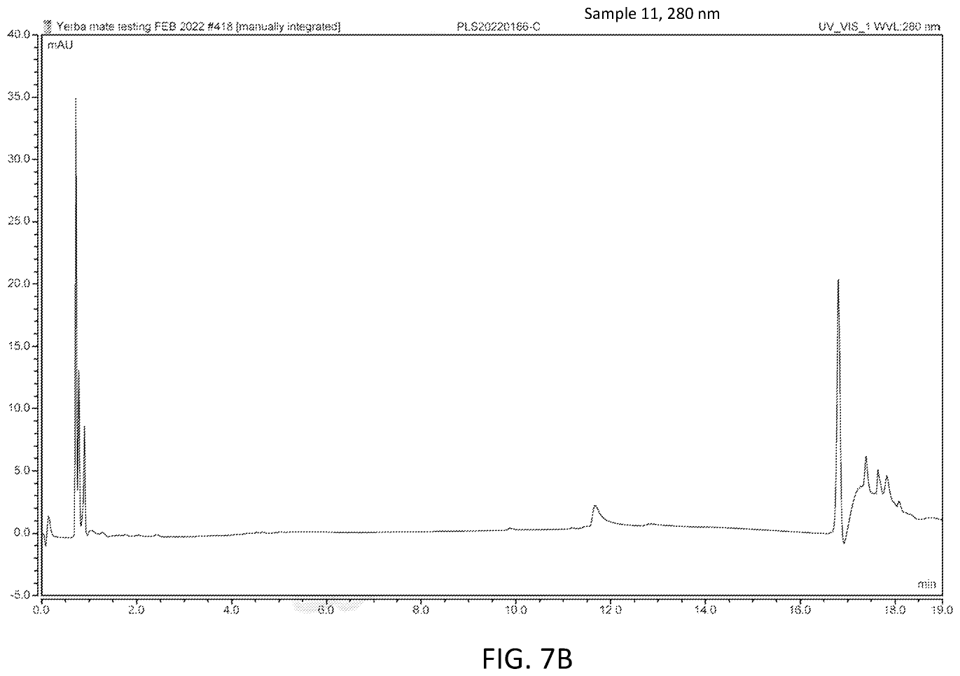

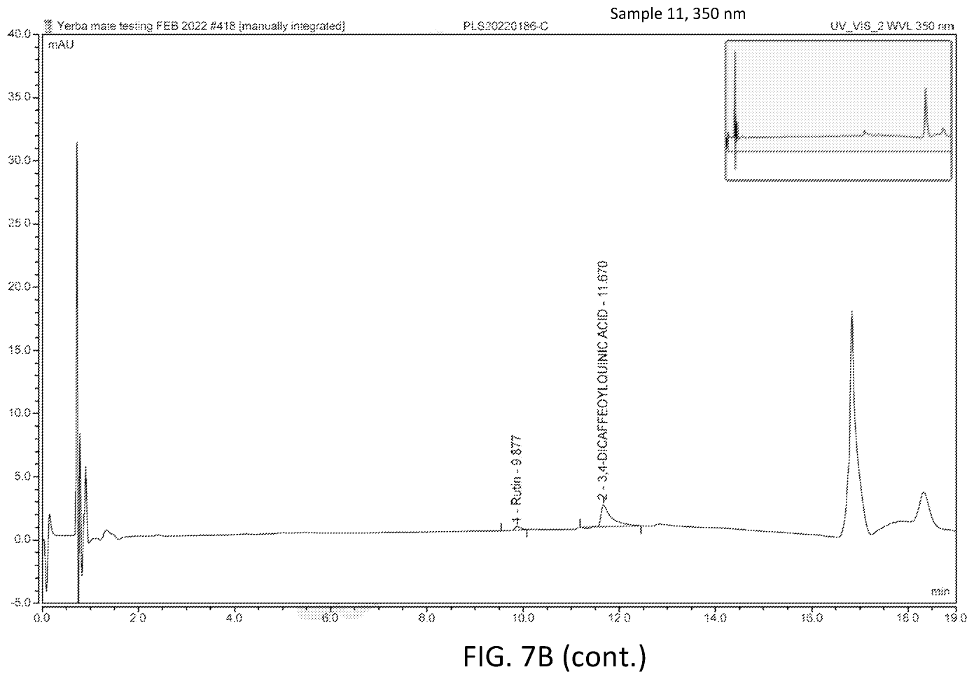

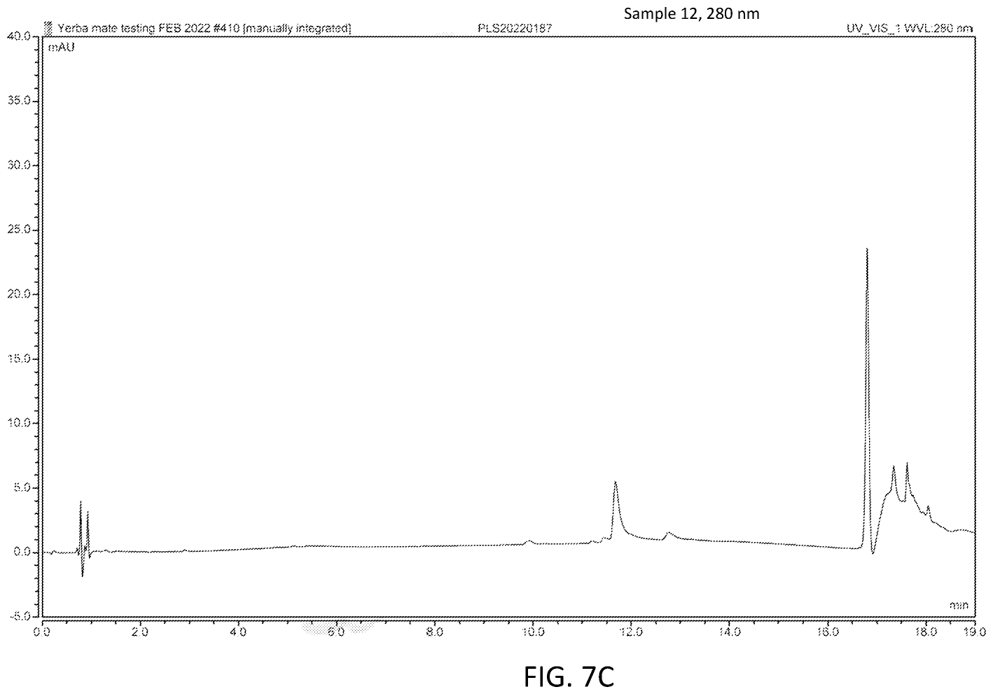

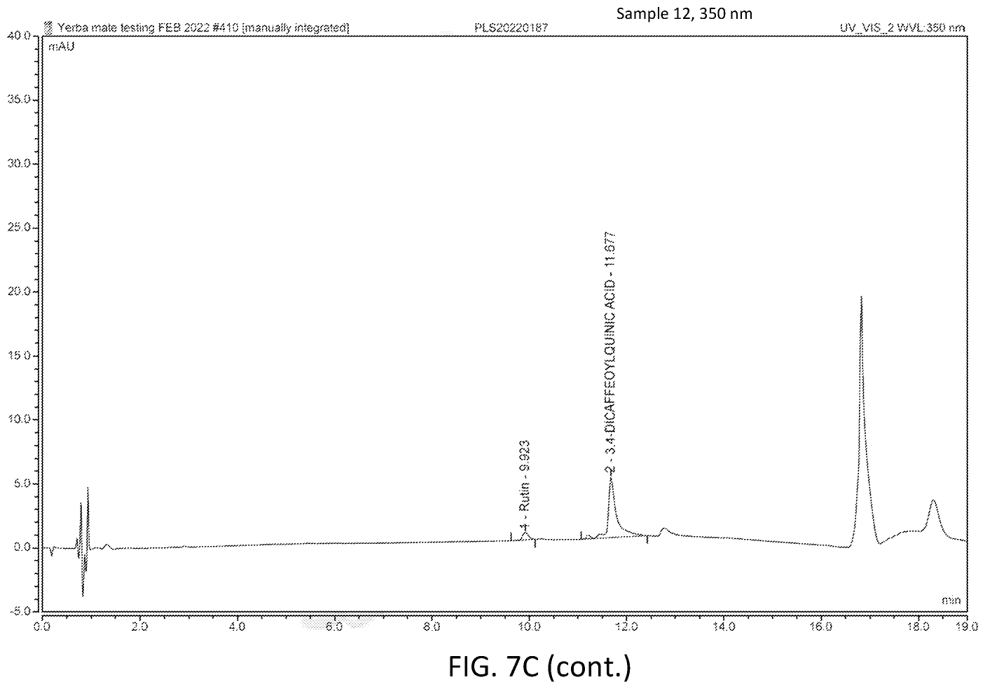

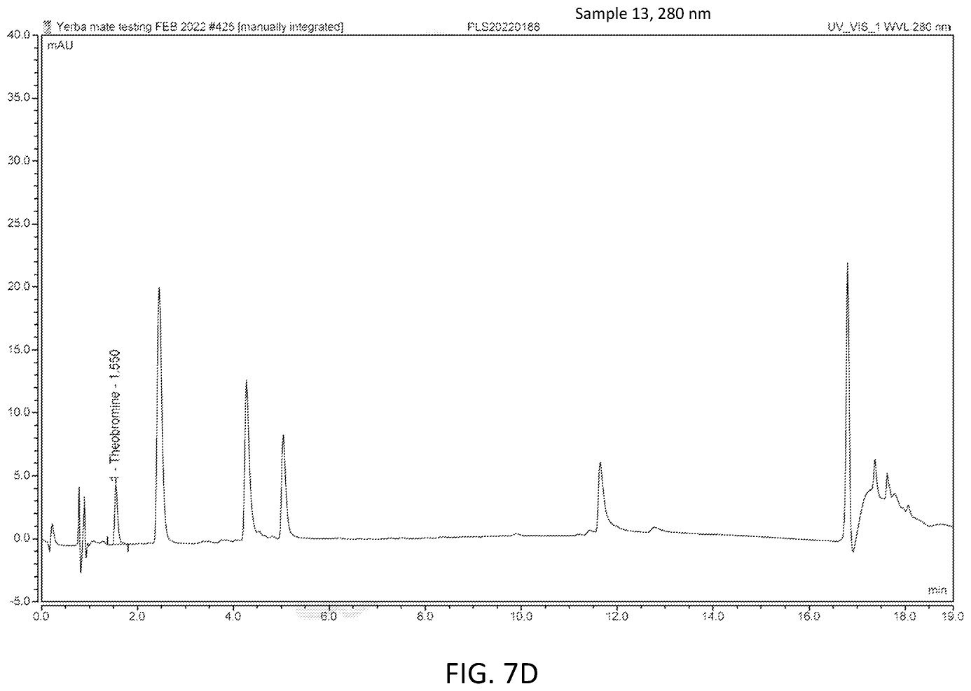

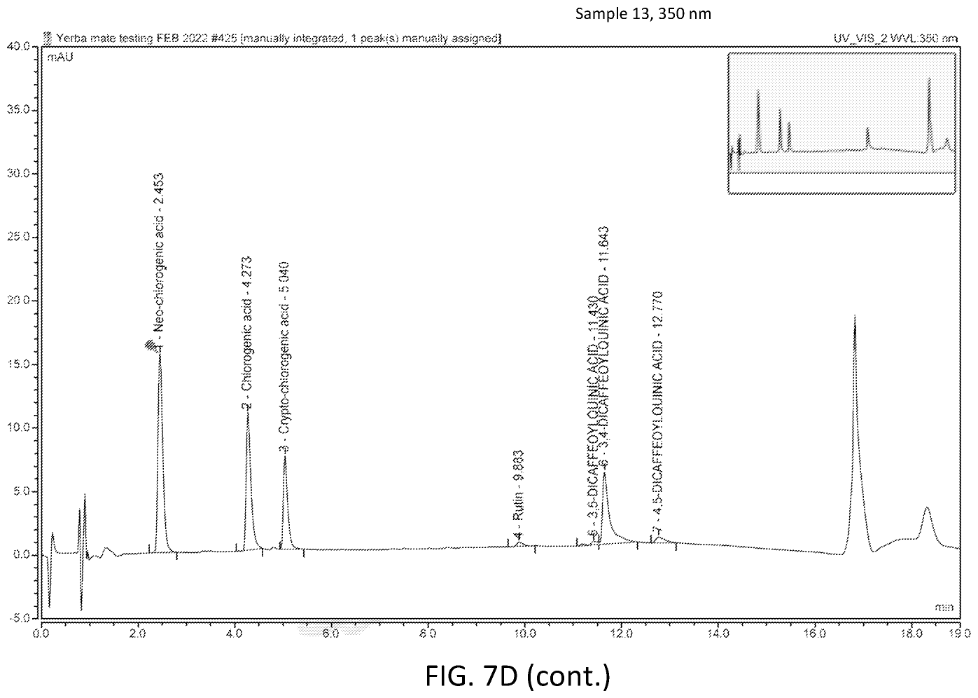

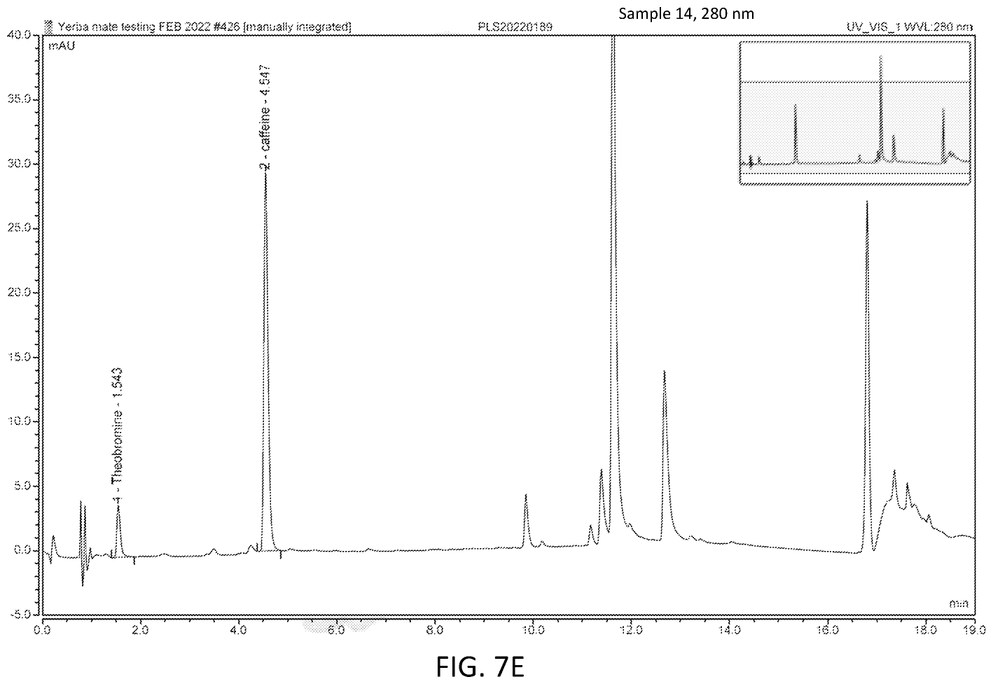

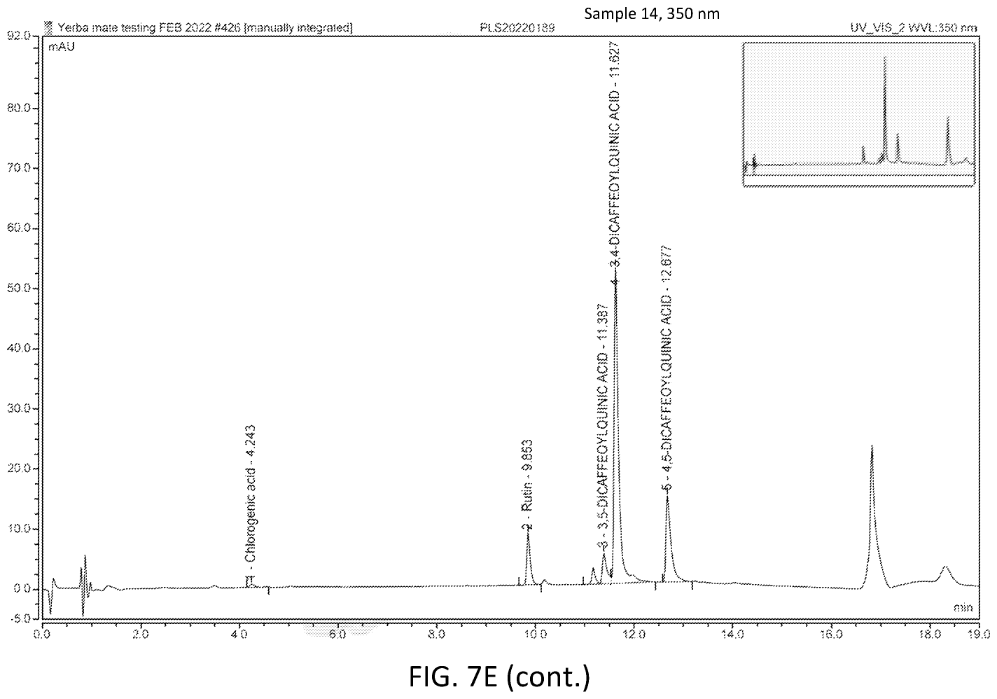

150 g crude extract solution (Sample 10) containing 11.2 g solid was loaded on the column. The solid content included bioactives, impurities, and NaDES ingredient. The concentration of this loading solution was 0.75%, and that was not NaDES because large amount of water was breaking up the hydrogen bond, but the individual components, chlorine chloride, and glucose were present in the solution. HPLC chromatograms of this Sample 10 are shown in FIG. 7A. All the bioactives were present in this sample.

First, Sample 10 was loaded on the column. During the loading process, 5.4 g solid (that is 48% of loading solution) was collected in effluent. HPLC chromatograms of effluent Sample 11 are shown in FIG. 7B, only negligible amount of bioactives were detected. After concentration, the fraction felt sticky, which meant NaDES s ingredients were present. This fraction was recycled.

The loaded column was sequentially washed by water, 20% v/v ethanol and 70% v/v ethanol and fractions Sample 12 (water fraction), Sample 13 (20% ethanol fraction), Sample 14 (70% ethanol fraction) were collected for mass balance and chemistry testing. HPLC chromatograms of Sample 12 are shown in FIG. 7C.

3.3 g solid, that is 30% of solid in crude extracts, was collected in water fraction Sample 12. HPLC chromatogram showed that a total of about 0.4 mg (0.3% in crude extracts) bioactives were collected in this fraction. After concentration, this fraction also felt sticky, which meant NaDESs ingredients were present. This fraction was also recycled.

In a 20% ethanol fraction Sample 13, 38% of bioactives in crude extracts were recovered. Remarkably, caffeine was completely removed, but theobromine was present and the purity was increased from 0.06% in crude extract to 2.44%; three chlorogenic acids isomers were all concentrated significantly. Chlorogenic acid was concentrated from 0.15% to 5.24%; neo-chlorogenic acid was concentrated from 0.22% to 7.64% and crypto-chlorogenic acid was concentrated from 0.09% to 3.14%. Dicaffeoylquinic acids were also present with higher concentration than those in crude extract. The processing yield of this fraction was 10% based on raw material. HPLC chromatograms of this Sample 13 are shown in FIG. 7D.

Caffeine and dicaffeoylquinic acids in crude extract were eluted by 70% ethanol in Sample 14. Comparing with caffeoylquinic acids, caffeine was non-polar, and was eluted in less polar solvents; and dicaffeoylquinic acids were less polar than caffeoylquinic acid because of space hinderance. HPLC chromatograms of this sample 14 are shown in FIG. 7E.

### Example 5

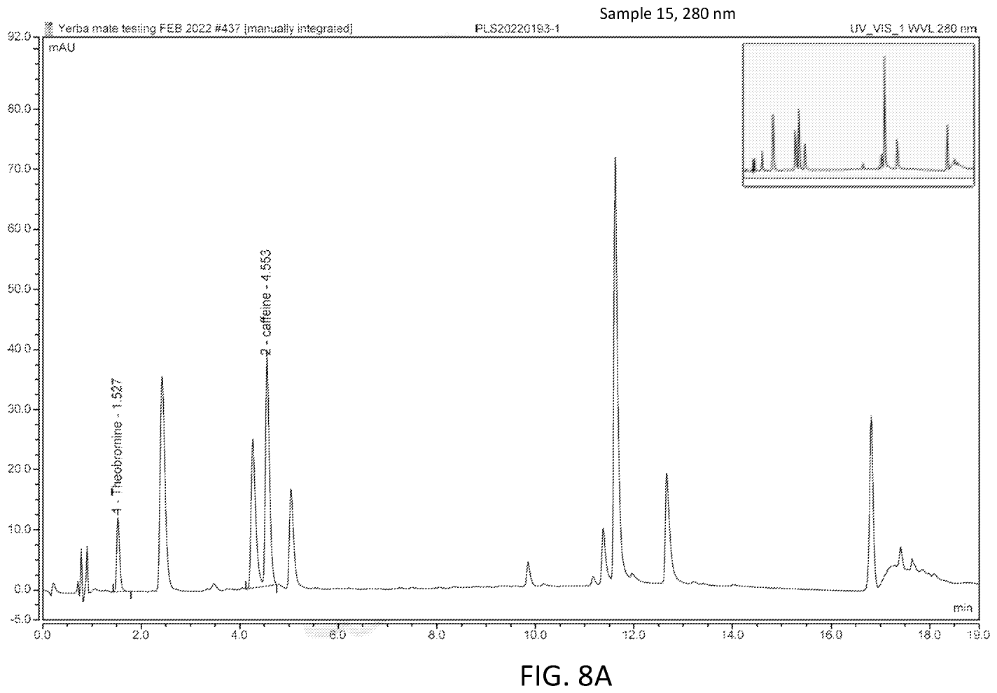

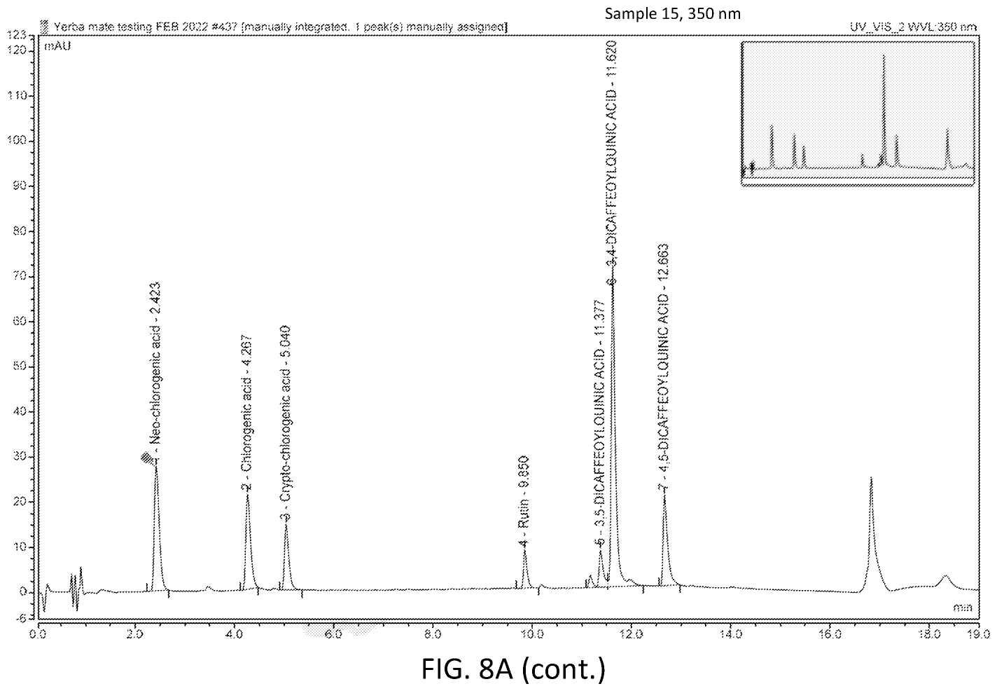

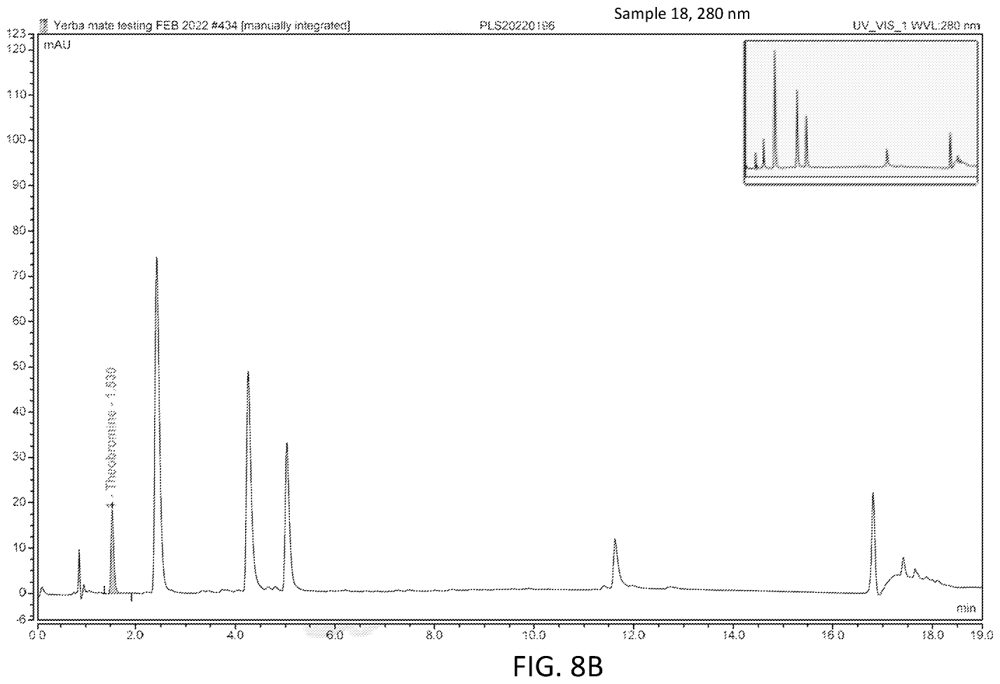

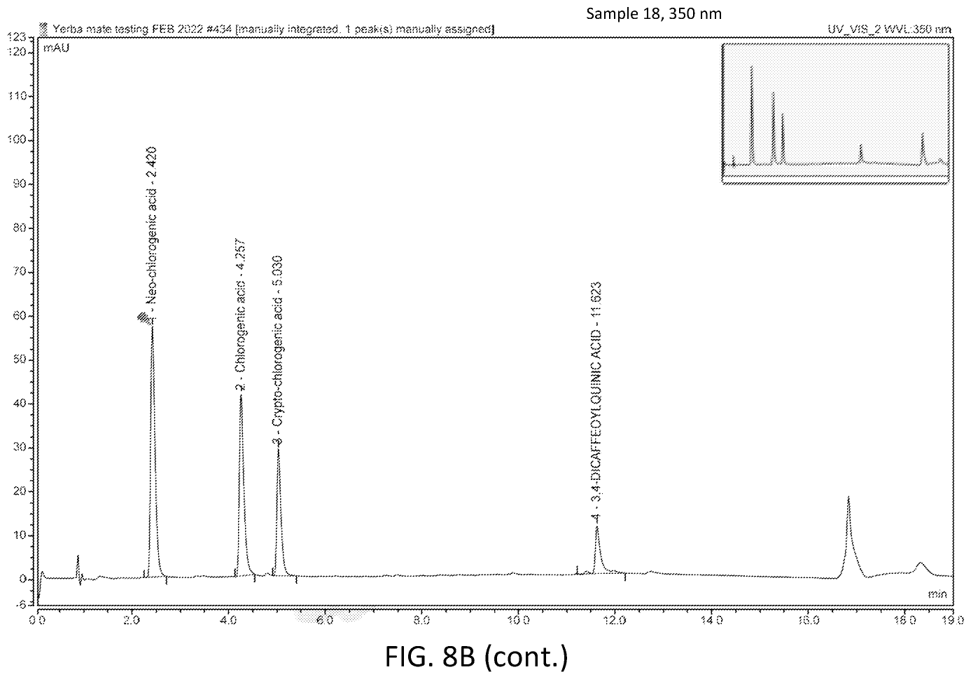

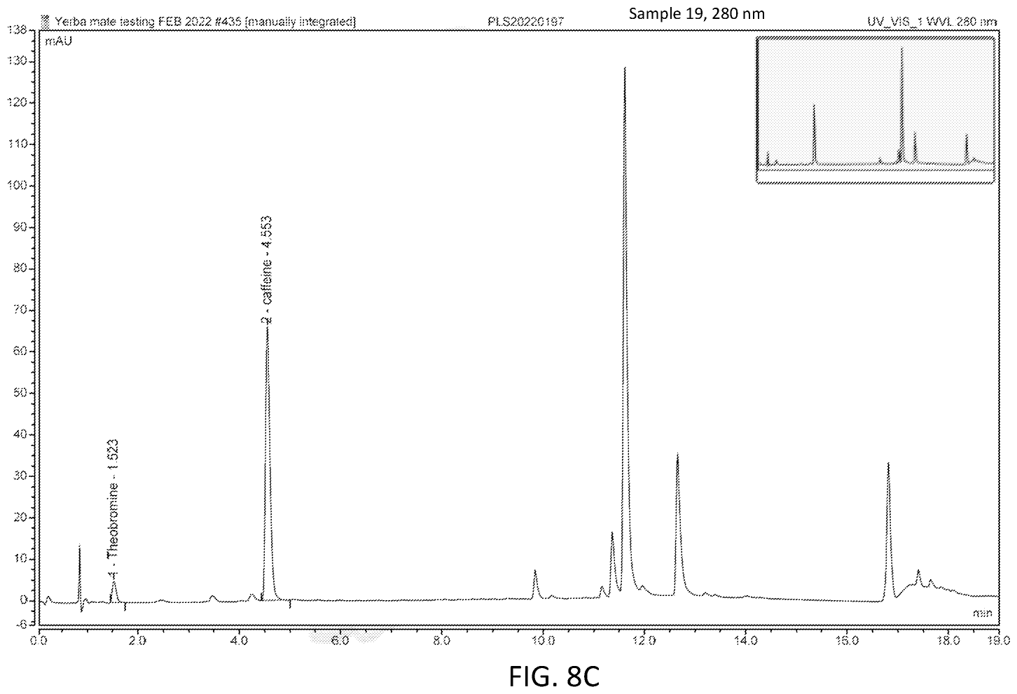

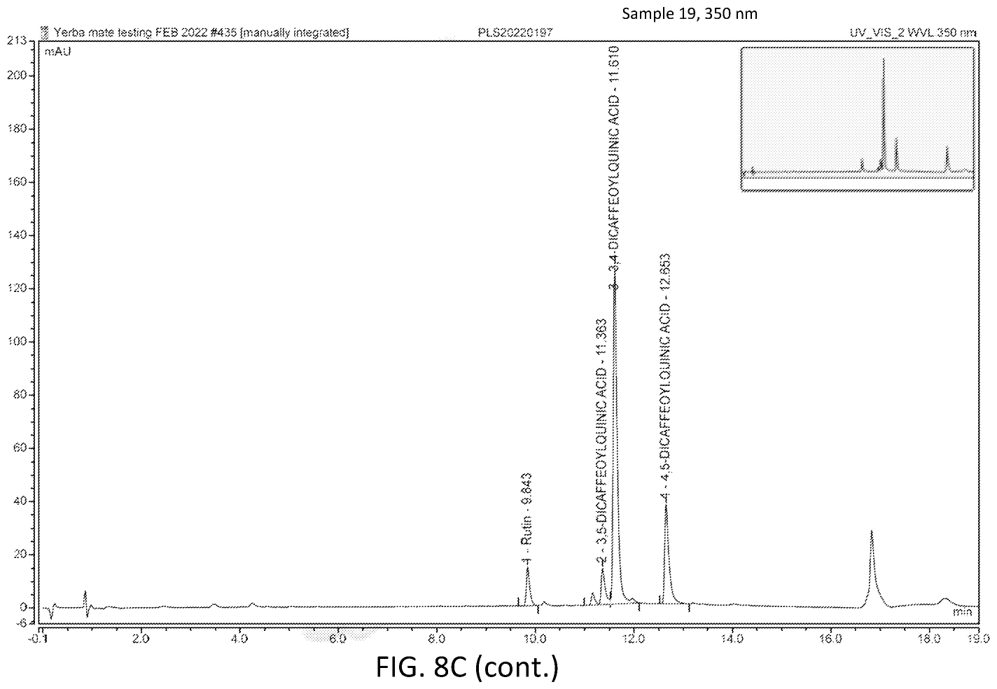

Based on the results above, the process for caffeine removal and NaDES recycling was also performed on a 1 L pilot scale. The results are shown in Table 7 and FIGS. 8A-8C.

TABLE 7
Results of gradient elution of yerba mate bioactives on 1 L column packed with the HPD200 resin
(A) The purity

| | | | | Neo- | | Crypto- | | 3,4- | 3,5- | 4,5- | |
|---|---|---|---|---|---|---|---|---|---|---|---|
| | | | | chloro- | Chloro- | chloro- | | dicaffe- | dicaffe- | dicaffe- | |
| | | Theo- | | genic | genic | genic | | oylquinic | oylquinic | oylquinic | Total |
| Sample | Description | bromine | Caffeine | acid | acid | acid | Rutin | acid | acid | acid | CQA |
| 15 | Crude | 0.07% | 0.09% | 0.17% | 0.12% | 0.07% | 0.02% | 0.03% | 0.21% | 0.07% | 0.68% |
| | extract | | | | | | | | | | |
| 18 | 20% EtOH | 2.37% | — | 7.81% | 5.65% | 3.38% | 0.01% | 0.15% | 1.11% | 0.25% | 18.36% |
| | elutes | | | | | | | | | | |
| 19 | 70% EtOH | 1.88% | 8.04% | 0.00% | 0.00% | 0.00% | 2.02% | 2.46% | 17.24% | 5.64% | 27.37% |
| | elutes | | | | | | | | | | |

(B) The weight of bioactives

| Sample | 15 | 16 | 17 | 18 | 19 |
|---|---|---|---|---|---|
| Description | Crude extract | Effluent | Water elutes | 20% EtOH elutes | 70% EtOH elutes |
| Total Solid (g) | 113.00 | 61.50 | 33.70 | 2.00 | 1.20 |
| Step processing rate | — | 54.4% | 29.8% | 1.8% | 1.1% |
| Total processing | — | — | — | 10.0% | 6.0% |
| yield | | | | | |
| Bioactives | 5.30% | 0.15% | 0.11% | 2.59% | 2.33% |
| processing yield | | | | | |
| Theobromine (mg) | 77.68 | — | — | 47.31 | 18.83 |
| Caffeine (mg) | 103.74 | — | — | — | 80.42 |
| Neo-chlorogenic | 187.59 | — | — | 156.14 | — |
| acid (mg) | | | | | |
| Chlorogenic acid | 137.49 | — | — | 112.99 | — |
| (mg) | | | | | |
| Crypto-chlorogenic | 80.67 | — | — | 67.67 | — |
| acid (mg) | | | | | |
| Rutin (mg) | 18.72 | — | — | 0.26 | 20.21 |
| 3,4-dicaffeoylquinic | 35.17 | — | — | 2.98 | 24.56 |
| acid | | | | | |
| 3,5-dicaffeoylquinic | 234.90 | 20.95 | 17.24 | 22.18 | 172.45 |
| acid | | | | | |
| 4,5-dicaffeoylquinic | 78.48 | 8.65 | 5.27 | 4.91 | 56.45 |
| acid | | | | | |

The process was successfully scaled up to 1 L column that was 10 times larger than small column. The scale-up showed repeatability and similar trend in results, as follows.

1500 ml of crude extracts solution (Sample 15) containing 113 g solid including 98 g NaDESs and 0.95 g measurable bioactives. Others were impurity extracted from yerba mate leaves. The crude extract (Sample 15) was loaded on column, 61.5 g solid and 33.7 g solid was collected in effluent (Sample 16) and water washing solution (Sample 17). NaDES was recovered in these fractions for recycling.

The content of caffeine in crude (Sample 15) was 0.09%; and the content of total caffeoylquinic acids (Total CQA) that combines all the caffeoylquinic acid and dicaffeoylquinic acid was 0.68%. HPLC chromatograms of this Sample 15 are shown in FIG. 8A. The content of bioactives in crude extract were low because of large amount of NaDES was present.

In 20% EtOH elutes (Sample 18), the content of caffeine was zero, but the content of Total CQA was 18.36%, which was 27-fold the amount present in crude extracts. HPLC chromatograms of this Sample 18 are shown in FIG. 8B. The processing yield of this fraction was 10%. In this fraction, in total CQA, more than 90% was caffeyolquinic acid.

In 70% EtOH elutes (Sample 19), the content of caffeine was 6.7% with 22.81% Total CQA. And in this fraction, more than 90% was dicaffeyolquinic acid. HPLC chromatograms of this Sample 19 are shown in FIG. 8C.

### Example 6

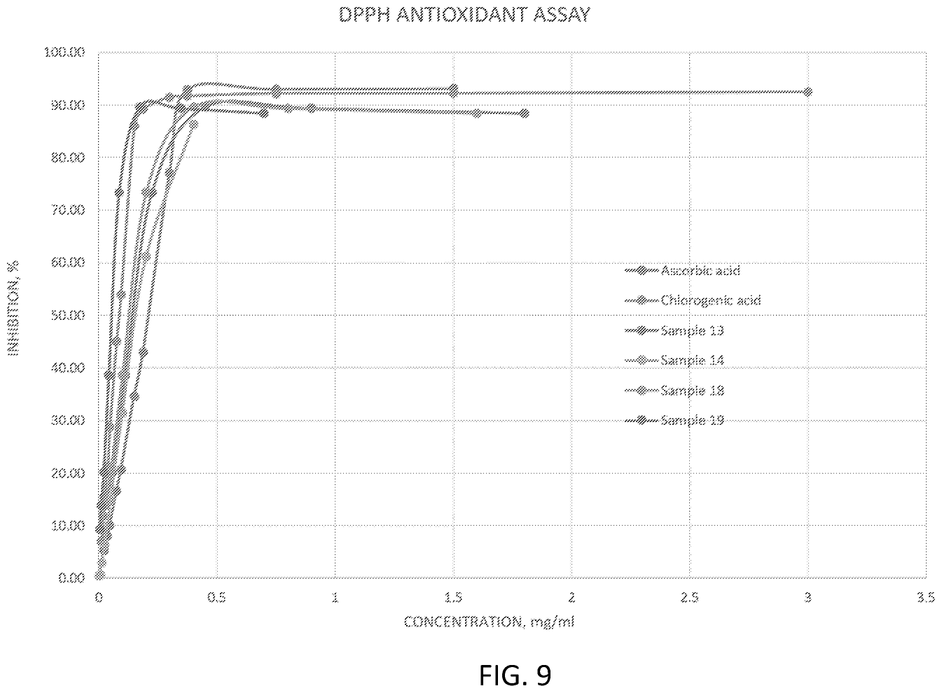

To evaluate the antioxidant activity of different fractions, an assay method based on the reduction of DPPH was performed. Pure chlorogenic acid and ascorbic acid were used as the positive control. The results of the assay are shown in FIG. 9 and the calculated IC₅₀ values are shown in Table 8.

TABLE 8
DPPH antioxidant assay IC₅₀ results

| Sample | IC₅₀, μg/ml |
|---|---|
| Chlorogenic Acid | 96.8 |
| Ascorbic Acid | 59.4 |
| Sample 13 | 107.3 |
| Sample 18 | 104.0 |
| Sample 14 | 142.1 |
| Sample 19 | 167.5 |

Remarkably, decaffeinated, caffeyolquinic acid enriched yerba mate extracts Sample 13 and Sample 18 showed the ICso value of 104-107 μg/ml, nearly equivalent to pure chlorogenic acid. In this fraction, in total caffeoylquinic acid content, greater than 90% were monoferoylquinic acids; interestingly, 70% ethanol elution fraction Sample 14 and Sample 19, the ICso values were 142-167 μg/ml even though they were higher in total caffeoylquinic acid contents. In this fraction, in total caffeoylquinic acid content, more than 90% were dicaffeoylquinic acids. The results suggested that dicaffeoylquinic acids have weaker antioxidant activity than caffeoylquinic acids isomers and caffeine did not contribute to antioxidant activity.

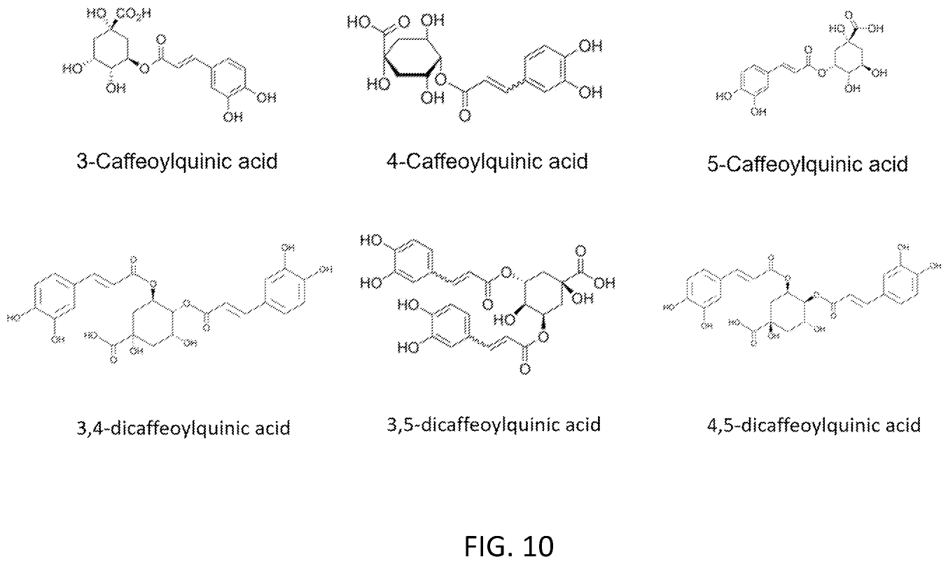

Polyphenols play multiple biological functions, and many of these functions have been attributed to their antioxidant activity (Pandey & Rizvi, 2009). Their structures are shown in FIG. 10. In the present study, caffeoylquinic acids (3-,4-,5-) possessed better antioxidative activities than dicaffeoylquinic acids (3,4-,3,5-,4,5-). Generally, the antioxidant activity was shown to increase with an increase in hydroxyl groups and to enhance the presence of ortho-hydroxyl groups attached to the aromatic ring. The dicaffeoylquinic acid has one more hydroxyl group or ortho-hydroxyl groups attached to the aromatic ring than the caffeoylquinic acid, which might be mainly responsible for exhibiting better antioxidant activities. However, surprisingly, the antioxidant activities of smaller size caffeoylquinic acids on the DPPH inhibition were higher than that of dicaffeoylquinic acids, indicating that the amount of hydroxyl groups is not the sole factor important for the protection against DPPH oxidation. This result implied that the antioxidant activities of dicaffeoylquinic acids were probably influenced by the position of esterification on the quinic moiety. It might be expected that there may exist a steric hindrance effect in the dicaffeoylquinic acids because of the greater distance, which can decrease the chemical bonding energy and stabilize its molecules relative to caffeoylquinic acids.

Together, these examples demonstrated that caffeine removed caffeoylquinic acid isomers (3-,4-,5-) concentrated fraction exhibited unexpectedly superior antioxidant activities, equivalent to pure caffeoylquinic acid and ascorbic acid. Caffeoylquinic acid concentrated fraction showed better DPPH inhibition than dicaffeoylquinic acid even though dicaffeoylquinic acid has more hydroxyl group or ortho-hydroxyl groups, suggesting that the amount of hydroxyl groups was not the sole factor important for the protection against DPPH oxidation, and the position of esterification on the quinic moiety of caffeoylquinic acid had great influence. The results suggest steric hindrance effects in the dicaffeoylquinic acids, resulting in their weaker antioxidant activities.

### Other Considerations

Headings are included herein for reference and to aid in locating various sections. These headings are not intended to limit the scope of the concepts described with respect thereto. Such concepts may have applicability throughout the entire specification.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents are based on available information and is not an admission as to their accuracy or correctness.

In the foregoing description, specific details are given to provide a thorough understanding of the examples. However, it will be understood by one of ordinary skill in the art that the examples may be practiced without these specific details.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the claims.

The methods disclosed herein comprise one or more steps or actions for achieving the described method. The method steps and/or actions may be interchanged with one another without departing from the scope of the present disclosure. In other words, unless a specific order of steps or actions is required for proper operation of the method that is being described, the order and/or use of specific steps and/or actions may be modified without departing from the scope of the present disclosure.

In at least some of the described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural or singular terms herein, those having skill in the art can translate from the plural to the singular or from the singular to the plural as is appropriate to the context or application. The various singular/plural permutations may be expressly set forth herein for sake of clarity.

The embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of the embodiments.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

### REFERENCES

Abbott, A. P., Capper, G., Davies, D. L., Rasheed, R. K., & Tambyrajah, V. (2003). Novel solvent properties of choline chloride/urea mixtures. Chemical Communications, 1, 70-71.
Abebe, M. W., Ntiamoah, R. A., & Kim, H. (2022). Alginate/chitosan bi-layer hydrogel as a novel tea bag with in-cup decaffeination. Reactive and Functional Polymers, 170, 105128.
Ahuja, S. (2002). CHROMATOGRAPHY AND SEPARATION SCIENCE.
Andrade, A., Sousa, C., Pedro, M., & Fernandes, M. (2018). Dangerous mistake: an accidental caffeine overdose. BMJ Case Reports, 2018, bcr2018224185.
Ashihara, H., & Suzuki, T. (2004). Distribution and biosynthesis of caffeine in plants. Frontiers in Bioscience, 9.
Banerjee, S., & Chatterjee, J. (2015). Efficient extraction strategies of tea (Camellia sinensis) biomolecules. Journal of Food Science and Technology, 52(6), 3158-3168.
Bermejo, D. V., Luna, P., Manic, M. S., Najdanovic-Visak, V., Reglero, G., & Fornari, T. (2013). Extraction of caffeine from natural matter using a bio-renewable agrochemical solvent. Food and Bioproducts Processing, 91(4), 303-309.
Bolton, S., & Null, G. (1981). Caffeine: Psychological effects, use and abuse. Orthomolecular Psychiatry, 10(3), 202-211.
Choi, Y. H., & Verpoorte, R. (2019). Green solvents for the extraction of bioactive compounds from natural products using ionic liquids and deep eutectic solvents. Current Opinion in Food Science, 26, 87-93.
Choung, M., Hwang, Y., Lee, M., Lee, J., Kang, S., & Jun, T. (2014). Comparison of extraction and isolation efficiency of catechins and caffeine from green tea leaves using different solvent systems. International Journal of Food Science & Technology, 49(6), 1572-1578.
da Silveira, T. F. F., Meinhart, A. D., de Souza, T. C. L., Cunha, E. C. E., de Moraes, M. R., & Godoy, H. T. (2017). Chlorogenic acids and flavonoid extraction during the preparation of yerba mate based beverages. Food Research International, 102, 348-354.
Dai, Y., Van Spronsen, J., Witkamp, G.-J., Verpoorte, R., & Choi, Y. H. (2013). Ionic liquids and deep eutectic solvents in natural products research: mixtures of solids as extraction solvents. Journal of Natural Products, 76(11), 2162-2173.
Dash, S. S., & Gummadi, S. N. (2006). Catabolic pathways and biotechnological applications of microbial caffeine degradation. Biotechnology Letters, 28(24), 1993-2002.
Davis, J. M., Zhao, Z., Stock, H. S., Mehl, K. A., Buggy, J., & Hand, G. A. (2003). Central nervous system effects of caffeine and adenosine on fatigue. American Journal of Physiology-Regulatory, Integrative and Comparative Physiology.
De Marco, I., Riemma, S., & Iannone, R. (2017). Supercritical carbon dioxide decaffeination process: A life cycle assessment study. Chemical Engineering Transactions, 57, 1699-1704.
Derry, C. J., Deny, S., & Moore, R. A. (2014). Caffeine as an analgesic adjuvant for acute pain in adults. Cochrane Database of Systematic Reviews, 12.
do Espirito Santo, A. T., Siqueira, L. M., Almeida, R. N., Vargas, R. M. F., do N Franceschini, G., Kunde, M. A., Cappellari, A. R., Morrone, F. B., & Cassel, E. (2021). Decaffeination of yerba mate by supercritical fluid extraction: Improvement, mathematical modelling and infusion analysis. The Journal of Supercritical Fluids, 168, 105096.
Dong, J.-J., Ye, J.-H., Lu, J.-L., Zheng, X.-Q., & Liang, Y.-R. (2011). Isolation of antioxidant catechins from green tea and its decaffeination. Food and Bioproducts Processing, 89(1), 62-66.
Du, H., Wang, H., Yu, J., Liang, C., Ye, W., & Li, P. (2012). Enrichment and purification of total flavonoid C-glycosides from Abrus mollis extracts with macroporous resins. Industrial & Engineering Chemistry Research, 51(21), 7349-7354.
Foskett, A., Ali, A., & Gant, N. (2009). Caffeine Enhances Cognitive Function and Skill Performance during Simulated Soccer Activity. International Journal of Sport Nutrition and Exercise Metabolism, 19(4), 410-423.
Friedman, M., & Jiirgens, H. S. (2000). Effect of pH on the Stability of Plant Phenolic Compounds. Journal of Agricultural and Food Chemistry, 48(6), 2101-2110.
Glade, M. J. (2010). Caffeine—Not just a stimulant. Nutrition, 26(10), 932-938.
Gokulakrishnan, S., Chandraraj, K., & Gummadi, S. N. (2005). Microbial and enzymatic methods for the removal of caffeine. Enzyme and Microbial Technology, 37(2), 225-232.
GREDEN, J. F. (1974). Anxiety or Caffeinism: A Diagnostic Dilemma. American Journal of Psychiatry, 131(10), 1089-1092.
Gullón, P., Gullón, B., Romaní, A., Rocchetti, G., & Lorenzo, J. M. (2020). Smart advanced solvents for bioactive compounds recovery from agri-food by-products: A review. Trends in Food Science & Technology, 101, 182-197.
Hayyan, A., Mjalli, F. S., AlNashef, I. M., Al-Wahaibi, Y. M., Al-Wahaibi, T., & Hashim, M. A. (2013). Glucose-based deep eutectic solvents: Physical properties. Journal of Molecular Liquids, 178, 137-141.
Heck, C. I., & Mejia, E. G. De. (2007). Yerba Mate Tea (Ilex paraguariensis): A Comprehensive Review on Chemistry, Health Implications, and Technological Considerations. Journal of Food Science, 72(9), R138-R151.
Heckman, M. A., Weil, J., & De Mejia, E. G. (2010). Caffeine (1,3,7-trimethylxanthine) in Foods: A Comprehensive Review on Consumption, Functionality, Safety, and Regulatory Matters. Journal of Food Science, 75(3), R77-R87.
Herz, R. S. (1999). Caffeine effects on mood and memory. Behaviour Research and Therapy, 37(9), 869-879.
Huang, Y., Feng, F., Jiang, J., Qiao, Y., Wu, T., Voglmeir, J., & Chen, Z.-G. (2017). Green and efficient extraction of rutin from tartary buckwheat hull by using natural deep eutectic solvents. Food Chemistry, 221, 1400-1405.
Jin, X., Liu, M., Chen, Z., Mao, R., Xiao, Q., Gao, H., & Wei, M. (2015). Separation and purification of epigallocatechin-3-gallate (EGCG) from green tea using combined macroporous resin and polyamide column chromatography. Journal of Chromatography B, 1002, 113-122.
Júnior, M. A. D., Borsoi, C., Hansen, B., & Catto, A. L. (2019). Evaluation of different methods for extraction of nanocellulose from yerba mate residues. Carbohydrate Polymers, 218, 78-86.
Kanda, H., Li, P., & Makino, H. (2013). Production of decaffeinated green tea leaves using liquefied dimethyl ether. Food and Bioproducts Processing, 91(4), 376-380.
Katz, S. N. (1987). Decaffeination of coffee. In Coffee (pp. 59-71). Springer.
Lack, E., & Seidlitz, H. (1993). Commercial scale decaffeination of coffee and tea using supercritical CO 2. In Extraction of natural products using near-critical solvents (pp. 101-139). Springer.
Lee, S., Park, M. K., Kim, K. H., & Kim, Y. (2007). Effect of supercritical carbon dioxide decaffeination on volatile components of green teas. Journal of Food Science, 72(7), S497-S502.
Li, A., Xuan, H., Sun, A., Liu, R., & Cui, J. (2016). Preparative separation of polyphenols from water-soluble fraction of Chinese propolis using macroporous absorptive resin coupled with preparative high performance liquid chromatography. Journal of Chromatography B, 1012, 42-49.
Li, C., Zheng, Y., Wang, X., Feng, S., & Di, D. (2011). Simultaneous separation and purification of flavonoids and oleuropein from Olea europaea L.(olive) leaves using macroporous resin. Journal of the Science of Food and Agriculture, 91(15), 2826-2834.
Li, H., Shi, J., Li, Y., Wang, C., Hou, G., Cong, W., & Zhao, F. (2019). Purification of spinosin from Ziziphi Spinosae Semen using macroporous resins followed by preparative high-performance liquid chromatography. Journal of Separation Science, 42(19), 3134-3140.
Li, J., & Chase, H. A. (2010). Development of adsorptive (non-ionic) macroporous resins and their uses in the purification of pharmacologically-active natural products from plant sources. Natural Product Reports, 27(10), 1493-1510.
Liang, H., Liang, Y., Dong, J., Lu, J., Xu, H., & Wang, H. (2007). Decaffeination of fresh green tea leaf (Camellia sinensis) by hot water treatment. Food Chemistry, 101(4), 1451-1456.
Lieberman, H. R., Wurtman, R. J., Emde, G. G., Roberts, C., & Coviella, I. L. G. (1987). The effects of low doses of caffeine on human performance and mood. Psychopharmacology, 92(3), 308-312.
Lin, L., Zhao, H., Dong, Y., Yang, B., & Zhao, M. (2012). Macroporous resin purification behavior of phenolics and rosmarinic acid from Rabdosia serra (MAXIM.) HARA leaf. Food Chemistry, 130(2), 417-424.
Linares, A. R., Hase, S. L., Vergara, M. L., & Resnik, S. L. (2010). Modeling yerba mate aqueous extraction kinetics: Influence of temperature. Journal of Food Engineering, 97(4), 471-477.
Liu, Y., Di, D., Bal, Q., Li, J., Chen, Z., Lou, S., & Ye, H. (2011). Preparative separation and purification of rebaudioside A from steviol glycosides using mixed-mode macroporous adsorption resins. Journal of Agricultural and Food Chemistry, 59(17), 9629-9636.
Liu, Z., Wang, J., Gao, W., Man, S., Wang, Y., & Liu, C. (2013). Preparative separation and purification of steroidal saponins in Paris polyphylla var. yunnanensis by macroporous adsorption resins. Pharmaceutical Biology, 51(7), 899-905.
Lomba, L., Zuriaga, E., & Giner, B. (2019). Solvents derived from biomass and their potential as green solvents. Current Opinion in Green and Sustainable Chemistry, 18, 51-56.
Lorist, M. M., & Tops, M. (2003). Caffeine, fatigue, and cognition. Brain and Cognition, 53(1), 82-94.
Lu, J.-L., Wu, M.-Y., Yang, X.-L., Dong, Z.-B., Ye, J.-H., Borthakur, D., Sun, Q.-L., & Liang, Y.-R. (2010). Decaffeination of tea extracts by using poly (acrylamide-co-ethylene glycol dimethylacrylate) as adsorbent. Journal of Food Engineering, 97(4), 555-562.
Ma, C., Tao, G., Lou, Z., Wang, H., Gu, X., Hu, L., & Yin, M. (2009). Preparative separation and purification of rosavin in Rhodiola rosea by macroporous adsorption resins. Separation and Purification Technology, 69(1), 22-28.
Marriott, B. M. (1994). Food Components to Enhance Performance: An evaluation of potential performance-enhancing food components for operational rations.
Mazzafera, P. (2002). Degradation of caffeine by microorganisms and potential use of decaffeinated coffee husk and pulp in animal feeding. Scientia Agricola, 59, 815-821.
Nanjundaiah, S., Bhatt, P., Rastogi, N. K., & Thakur, M. S. (2016). Response surface optimization for decaffeination and theophylline production by Fusarium solani. Applied Biochemistry and Biotechnology, 178(1), 58-75.
Nawrot, P., Jordan, S., Eastwood, J., Rotstein, J., Hugenholtz, A., & Feeley, M. (2003). Effects of caffeine on human health. Food Additives & Contaminants, 20(1), 1-30.
Nojima, T., Naito, H., Kosaki, Y., Osako, T., Tanaka, K., Murata, A., & Nakao, A. (2019). Caffeine Intoxication in Pregnancy; a case Report. Archives of Academic Emergency Medicine, 7(1), e67-e67.
Ogita, S., Uefuji, H., Yamaguchi, Y., Koizumi, N., & Sano, H. (2003). Producing decaffeinated coffee plants. Nature, 423(6942), 823.
Oni, B. A., Sanni, S. E., Dahunsi, S. O., & Egere, B. C. (2022). Decaffeination of wastewater using activated carbon produced from velvet tamarind-pericarp (Dialium Guineense). International Journal of Phytoremediation, 24(4), 394-408.
Ozel, N., & Elibol, M. (2021). A review on the potential uses of deep eutectic solvents in chitin and chitosan related processes. Carbohydrate Polymers, 262, 117942.
Pandey, K. B., & Rizvi, S. I. (2009). Plant Polyphenols as Dietary Antioxidants in Human Health and Disease. Oxidative Medicine and Cellular Longevity, 2, 897484.
Park, H. S., Im, N. G., & Kim, K. H. (2012). Extraction behaviors of caffeine and chlorophylls in supercritical decaffeination of green tea leaves. LWT-Food Science and Technology, 45(1), 73-78.
Park, H. S., Lee, H. J., Shin, M. H., Lee, K.-W., Lee, H., Kim, Y.-S., Kim, K. 0., & Kim, K. H. (2007). Effects of cosolvents on the decaffeination of green tea by supercritical carbon dioxide. Food Chemistry, 105(3), 1011-1017.
Peker, H., Srinivasan, M. P., Smith, J. M., & McCoy, B. J. (1992). Caffeine extraction rates from coffee beans with supercritical carbon dioxide. AIChE Journal, 38(5), 761-770.
Pietsch, A. (2017). Decaffeination—Process and quality. In The craft and science of coffee (pp. 225-243). Elsevier.
Plotka-Wasylka, J., De la Guardia, M., Andruch, V., & Vilková, M. (2020). Deep eutectic solvents vs ionic liquids: Similarities and differences. Microchemical Journal, 159, 105539.
Quintero-Jaramillo, J. A., Carrero-Mantilla, J. I., & Sanabria-González, N. R. (2021). A Review of Caffeine Adsorption Studies onto Various Types of Adsorbents. The Scientific World Journal, 2021, 9998924.
Ramalakshmi, K., & Raghavan, B. (1999). Caffeine in coffee: its removal. Why and how? Critical Reviews in Food Science and Nutrition, 39(5), 441-456.
Ranheim, T., & Halvorsen, B. (2005). Coffee consumption and human health—beneficial or detrimental?—Mechanisms for effects of coffee consumption on different risk factors for cardiovascular disease and type 2 diabetes mellitus. Molecular Nutrition & Food Research, 49(3), 274-284.
Reis, J. P., Loria, C. M., Steffen, L. M., Zhou, X., Van Horn, L., Siscovick, D. S., Jacobs Jr, D. R., & Carr, J. J. (2010). Coffee, decaffeinated coffee, caffeine, and tea consumption in young adulthood and atherosclerosis later in life: the CARDIA study. Arteriosclerosis, Thrombosis, and Vascular Biology, 30(10), 2059-2066.
Roy, G. M. (1994). Activated carbon applications in the food and pharmaceutical industries. CRC press.
Ruesgas-Ramón, M., Figueroa-Espinoza, M. C., & Durand, E. (2017). Application of Deep Eutectic Solvents (DES) for Phenolic Compounds Extraction: Overview, Challenges, and Opportunities. Journal of Agricultural and Food Chemistry, 65(18), 3591-3601.
Saloko, S., Sulastri, Y., & Wahyuni, S. (2020). The application of activated carbon from coconut shell and zeolite as adsorbents on coffee decaffeination using the Swiss Water Process (SWP). IOP Conference Series: Earth and Environmental Science, 443(1), 12067.
Samoggia, A., & Rezzaghi, T. (2021). The Consumption of Caffeine-Containing Products to Enhance Sports Performance: An Application of an Extended Model of the Theory of Planned Behavior. In Nutrients (Vol. 13, Issue 2).
San Juan, A. F., López-Samanes, Á., Jodra, P., Valenzuela, P. L., Rueda, J., Veiga-Herreros, P., Pérez-López, A., & Domínguez, R. (2019). Caffeine Supplementation Improves Anaerobic Performance and Neuromuscular Efficiency and Fatigue in Olympic-Level Boxers. In Nutrients (Vol. 11, Issue 9).
Shafie, M. H., Yusof, R., & Gan, C.-Y. (2019). Synthesis of citric acid monohydrate-choline chloride based deep eutectic solvents (DES) and characterization of their physicochemical properties. Journal of Molecular Liquids, 288, 111081.
Shiono, T., Yamamoto, K., Yotsumoto, Y., Kawai, J., Imada, N., Hioki, J., Naganuma, H., Eguchi, T., Kurihara, M., & Yoshida, A. (2017). Selective decaffeination of tea extracts by montmorillonite. Journal of Food Engineering, 200, 13-21.
Smith, G. A. (1988). Caffeine reduction as an adjunct to anxiety management. British Journal of Clinical Psychology, 27(3), 265-266.
Sun, Q.-L., Hua, S., Ye, J.-H., Lu, J.-L., Zheng, X.-Q., & Liang, Y.-R. (2010). Decaffeination of green tea by supercritical carbon dioxide. Journal of Medicinal Plants Research, 4(12), 1161-1168.
Takao, T., Kitatani, F., Watanabe, N., Yagi, A., & Sakata, K. (1994). A Simple Screening Method for Antioxidants and Isolation of Several Antioxidants Produced by Marine Bacteria from Fish and Shellfish. Bioscience, Biotechnology, and Biochemistry, 58(10), 1780-1783.
Turnbull, C. H. S. and D., Dolnicar, S., Chapple, A., Adorno, T., Horkheimer, M., Negus, K., Pickering, M., Leckenby, J., & Li, H. (2004). Scholar (11). In Why We Need the Journal of Interactive Advertising (Vol. 10, Issue 10, pp. 349-383).
Vanda, H., Dai, Y., Wilson, E. G., Verpoorte, R., & Choi, Y. H. (2018). Green solvents from ionic liquids and deep eutectic solvents to natural deep eutectic solvents. Comptes Rendus Chimie, 21(6), 628-638.
Villanueva-Bermejo, D., Reglero, G., & Fornari, T. (2017). Recent advances in the processing of green tea biomolecules using ethyl lactate. A review. Trends in Food Science & Technology, 62, 1-12.
Vuong, Q. V, & Roach, P. D. (2014). Caffeine in green tea: its removal and isolation. Separation & Purification Reviews, 43(2), 155-174.
Ward, N., Whitney, C., Avery, D., & Dunner, D. (1991). The analgesic effects of caffeine in headache. Pain, 44(2), 151-155.
Wikoff, D., Welsh, B. T., Henderson, R., Brorby, G. P., Britt, J., Myers, E., Goldberger, J., Lieberman, H. R., O'Brien, C., & Peck, J. (2017). Systematic review of the potential adverse effects of caffeine consumption in healthy adults, pregnant women, adolescents, and children. Food and Chemical Toxicology, 109, 585-648.
Wolde, T. (2014). Effects of caffeine on health and nutrition: A Review. Food Science and Quality Management, 30, 59-65.
Xiong, Q., Zhang, Q., Zhang, D., Shi, Y., Jiang, C., & Shi, X. (2014). Preliminary separation and purification of resveratrol from extract of peanut (Arachis hypogaea) sprouts by macroporous adsorption resins. Food Chemistry, 145, 1-7.
Ye, J. H., Jin, J., Liang, H. L., Lu, J. L., Du, Y. Y., Zheng, X. Q., & Liang, Y. R. (2009). Using tea stalk lignocellulose as an adsorbent for separating decaffeinated tea catechins. Bioresource Technology, 100(2), 622-628.
Zabot, G. L. (2020). Decaffeination using supercritical carbon dioxide. In Green Sustainable Process for Chemical and Environmental Engineering and Science (pp. 255-278). Elsevier.
Zhang, L., Wu, T., Xiao, W., Wang, Z., Ding, G., & Zhao, L. (2018). Enrichment and purification of total ginkgo flavonoid 0-glycosides from Ginkgo biloba extract with macroporous resin and evaluation of anti-inflammation activities in vitro. Molecules, 23(5), 1167.
Zhang, Q.-W., Lin, L.-G., & Ye, W.-C. (2018). Techniques for extraction and isolation of natural products: A comprehensive review. Chinese Medicine, 13(1), 1-26.
Zhang, W.-Y. (2001). A benefit-risk assessment of caffeine as an analgesic adjuvant. Drug Safety, 24(15), 1127-1142.
Zhang, W., Liu, X., Fan, H., Zhu, D., Wu, X., Huang, X., & Tang, J. (2016). Separation and purification of alkaloids from Sophora flavescens Ait. by focused microwave-assisted aqueous two-phase extraction coupled with reversed micellar extraction. Industrial Crops and Products, 86, 231-238.
Zhang, Y., Jiao, J., Liu, C., Wu, X., & Zhang, Y. (2008). Isolation and purification of four flavone C-glycosides from antioxidant of bamboo leaves by macroporous resin column chromatography and preparative high-performance liquid chromatography. Food Chemistry, 107(3), 1326-1336.
Zosel, K. (1978). Separation with supercritical gases: practical applications. Angewandte Chemie International Edition in English, 17(10), 702-709.

What is claimed is:

1. A method of extracting a bioactive compound from a plant material, the method comprising:
obtaining a plant material;
mixing the plant material with a natural deep eutectic solvent (NaDES);
obtaining a crude extract solution comprising extracts of the plant material, the bioactive compound, and NaDES;
loading the crude extract solution onto a macroporous resin; and
recovering plant extract from the macroporous resin, wherein the bioactive compound and NaDES have been removed from the plant extract.

2. The method of claim 1, wherein the bioactive compound is caffeine.

3. The method of claim 1, wherein the plant material is obtained from yerba mate.

4. The method of claim 1, wherein the NaDES comprises choline chloride (ChCl) and glucose (GLU).

5. The method of claim 4, wherein the choline chloride and glucose are present in a ratio of about 2:1.

6. The method of claim 1, wherein the NaDES comprises water present in an amount ranging from 10%-50% by weight.

7. The method of claim 6, wherein the NaDES comprises water present in an amount of about 30% by weight.

8. The method of claim 1, further comprising diluting the crude extract with water before applying the crude extract to the macroporous resin.

9. The method of claim 8, wherein the dilution is about a 10× dilution.

10. The method of claim 1, wherein the recovered plant extract is enriched for caffeoylquinic acids.

11. The method of claim 1, wherein the recovered plant extract contains less than 0.05% caffeine by weight.

12. The method of claim 1, wherein the recovered plant extract contains less than 0.01% caffeine by weight.

13. The method of claim 1, wherein the macroporous resin comprises a non-polar macroporous resin.

14. The method of claim 1, wherein the macroporous resin comprises HDP200.

15. The method of claim 1, wherein the macroporous resin is in a column.

16. The method of claim 1, wherein recovering the plant extract comprises eluting the plant extract from the macroporous resin.

17. The method of claim 16, wherein the plant extract is eluted with a solution comprising ethanol in an amount ranging from 10% to 30% by volume.

18. The method of claim 17, wherein the solution comprises about 20% ethanol by volume.

19. The method of claim 1, further comprising recovering NaDES from the macroporous resin.

20. The method of claim 19, wherein recovering NaDES from the macroporous resin comprises washing the macroporous resin with water, capturing effluent and water, and drying the captured effluent and water to recover the NaDES.
